(19) United States
(12) Patent Application Publication
(10) Pub. No.: US 2009/0082395 A1
(43) Pub. Date: Mar. 26, 2009

(54) SOLUBLE EPOXIDE HYDROLASE INHIBITORS

(75) Inventor: Richard D. Gless, JR., Oakland, CA (US)

Correspondence Address: FOLEY & LARDNER LLP, 975 PAGE MILL ROAD, PALO ALTO, CA, 94304, US

(73) Assignee: Arete Therapeutics, Inc.

(21) Appl. No.: 12/207,413

(22) Filed: Sep. 9, 2008

Related U.S. Application Data
(60) Provisional application No. 60/971,508, filed on Sep. 11, 2007.

(51) Int. Cl. A61K 31/47; C07D 215/38; A61K 31/357; C07D 319/16; C07D 317/46; A61K 31/4184; C07D 235/06; C07D 209/08; C07D 231/54; C07D 263/56; C07D 277/64; A61K 31/4045; A61K 31/416; A61K 31/423; A61K 31/428; A61P 9/00; A61P 9/12

(52) U.S. Cl. 514/313; 546/171; 514/452; 549/366; 549/439; 514/466; 514/394; 548/309.7; 548/503; 548/362.5; 548/217; 548/178; 514/415; 514/403; 514/375; 514/367

(57) ABSTRACT

Disclosed are amide, thioamide, urea and thiourea compounds and compositions that inhibit soluble epoxide hydrolase (sEH), methods for preparing the compounds and compositions, and methods for treating patients with such compounds and compositions. The compounds, compositions, and methods are useful for treating a variety of sEH mediated diseases, including hypertensive, cardiovascular, inflammatory, and diabetic-related diseases.

### CROSS REFERENCE TO RELATED APPLICATION

This application claims the benefit under 35 U.S.C. 119(e) of U.S. Provisional Application No. 60/971,508, filed Sep. 11, 2007, which is incorporated by reference in its entirety into this application.

### BACKGROUND OF THE INVENTION

1. Field of the Invention

This invention relates to the field of pharmaceutical chemistry. Provided herein are amide, thioamide, urea, and thiourea compounds that inhibit soluble epoxide hydrolase (sEH), pharmaceutical compositions containing such compounds, methods for preparing the compounds and formulations, and methods for treating patients with such compounds and compositions. The compounds, compositions, and methods are useful for treating a variety of sEH mediated diseases, including hypertensive, cardiovascular, inflammatory, metabolic syndrome, and diabetic-related diseases.

2. State of the Art

The arachidonate cascade is a ubiquitous lipid signaling cascade in which arachidonic acid is liberated from the plasma membrane lipid reserves in response to a variety of extra-cellular and/or intra-cellular signals. The released arachidonic acid is then available to act as a substrate for a variety of oxidative enzymes that convert arachidonic acid to signaling lipids that play critical roles in inflammation and other disease conditions. Disruption of the pathways leading to the lipids remains an important strategy for many commercial drugs used to treat a multitude of inflammatory disorders. For example, non-steroidal anti-inflammatory drugs (NSAIDs) disrupt the conversion of arachidonic acid to prostaglandins by inhibiting cyclooxygenases (COX1 and COX2). New asthma drugs, such as SINGULAIR™ disrupt the conversion of arachidonic acid to leukotrienes by inhibiting lipoxygenase (LOX).

Certain cytochrome P450-dependent enzymes convert arachidonic acid into a series of epoxide derivatives known as epoxyeicosatrienoic acids (EETs). These EETs are particularly prevalent in the vascular endothelium (cells that make up arteries and vascular beds), kidney, and lung. In contrast to many of the end products of the prostaglandin and leukotriene pathways, the EETs have a variety of anti-inflammatory and anti-hypertensive properties and are known to be potent vasodilators and mediators of vascular permeability.

While EETs have potent effects in vivo, the epoxide moiety of the EETs is rapidly hydrolyzed into the less active dihydroxyeicosatrienoic acid (DHET) form by an enzyme called soluble epoxide hydrolase (sEH). Inhibition of sEH has been found to significantly reduce blood pressure in hypertensive animals (see, e.g., Yu et al. Circ. Res. 87:992-8 (2000) and Sinal et al. J. Biol. Chem. 275:40504-10 (2000)), to reduce the production of proinflammatory nitric oxide (NO), cytokines, and lipid mediators, and to contribute to inflammatory resolution by enhancing lipoxin A₄ production in vivo (see Schmelzer et al. Proc. Nat'l Acad. Sci. USA 102(28):9772-7 (2005)).

Various small molecule compounds have been found to inhibit sEH and elevate EET levels (Morisseau et al. Annu. Rev. Pharmacol. Toxicol. 45:311-33 (2005)). The availability of more potent compounds capable of inhibiting sEH and its inactivation of EETs would be highly desirable for treating a wide range of disorders that are mediated by conversion of sEH to EET's including inflammation and hypertension.

### SUMMARY OF THE INVENTION

This invention relates to compounds and their pharmaceutical compositions, to their preparation, and to their uses for treating diseases mediated by soluble epoxide hydrolase (sEH). In accordance with one aspect of the invention, provided are compounds of Formula (I) or a pharmaceutically acceptable salt thereof:

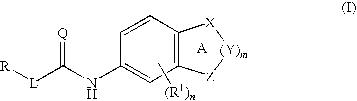

wherein:
A is an optionally substituted cycloalkyl, optionally substituted heterocyclic or optionally substituted heteroaryl ring fused with the benzene ring;
each of X, Y and Z are independently selected from the group consisting of —CR³═, —C(R⁴)(R⁵)—, —C(═O)—, —C(═S)—, —N═, —NR⁶—, —O—, —S—, —SO—, and —SO₂—, provided that at least one of X, Y and Z is selected from the group consisting of —C(═O)—, —C(═S)—, —N═, —NR⁶—, —O—, —S—, —SO—, and —SO₂—; and provided that X and Z are not both —C(═O)— and that A is not substituted with four fluoro;
   wherein
   each R³, R⁴ and R⁵ is independently selected from the group consisting of R⁶, halo, alkoxy, substituted alkoxy, acyloxy, carboxyl ester, acylamino, alkylamino, aminocarbonylamino, aminocarbonyloxy, aminosulfonylamino, (substituted sulfonyl)amino, (substituted sulfonyl)aminocarbonyl, (carboxyl ester)amino;
   R⁶ is independently selected from the group consisting of hydrogen, alkyl, substituted alkyl, alkenyl, substituted alkenyl, alkynyl, substituted alkynyl, heteroaryl, heterocyclyl, haloalkyl, acyl, aminosulfonyl, and aminocarbonyl;
m is 1, 2 or 3;
Q is O or S;
L is a covalent bond, —NH—, or —CR′R″— where R′ and R″ are independently H or alkyl or R′ and R″ together form a C₃-C₆ cycloalkyl ring;
each R¹ is independently selected from the group consisting of alkyl, cyano, halo, and haloalkyl;
n is 0, 1, 2, or 3;
R is selected from the group consisting of C₆₋₁₀ cycloalkyl, substituted C₆₋₁₀ cycloalkyl and

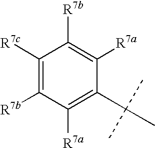

wherein each R^{7a} is independently hydrogen or halo;
each of R^{7b} and R^{7c} is independently selected from the group consisting of hydrogen, halo, alkyl, acyl, acyloxy, carboxyl ester, acylamino, alkylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, alkylsulfonyl and haloalkylsulfonyl; or R^{7c} and one of R^{7b} together form a heterocycloalkyl ring fused with the phenyl; provided that if one of R^{7b} is hydrogen and the other R^{7b} is halo, then R^{7c} and is not the same halo;
with the proviso that when L is —NH—, m is 1, and X, Y and Z with the phenyl ring define an indole or benzimidazole, then at least one of X, Y, or Z is selected from —NR⁶— and —CR³═, where R⁶ or R³ is not hydrogen; and
with the proviso that Formula (I) is not:

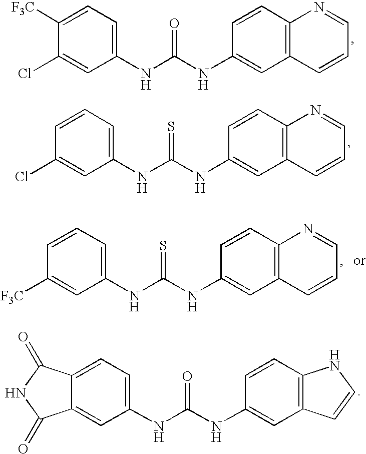

In another embodiment, provided are compounds of Formula (II), or a pharmaceutically acceptable salt thereof:

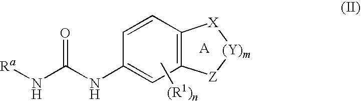

wherein:
A is an optionally substituted cycloalkyl, optionally substituted heterocyclic or optionally substituted heteroaryl ring fused with the benzene ring;
each of X, Y and Z are independently selected from the group consisting of —CR³═, —C(R⁴)(R⁵)—, —C(═O)—, —C(═S)—, ═N—, —NR⁶—, —O—, —S—, —SO—, and —SO₂—, provided that at least one of X, Y and Z is selected from the group consisting of —C(═O)—, —C(═S)—, —N═, —NR⁶—, —O—, —S—, —SO—, and —SO₂—; and provided that X and Z are not both —C(═O)— and that A is not substituted with four fluoro;
   wherein
   each R³, R⁴ and R⁵ is independently selected from the group consisting of R⁶, halo, alkoxy, substituted alkoxy, acyloxy, carboxyl ester, acylamino, alkylamino, aminocarbonylamino, aminocarbonyloxy, aminosulfonylamino, (substituted sulfonyl)amino, (substituted sulfonyl)aminocarbonyl, (carboxyl ester)amino;
   R⁶ is independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclyl, substituted heterocyclyl, haloalkyl, acyl, aminosulfonyl, and aminocarbonyl;
m is 1, 2 or 3;
each R¹ is independently selected from the group consisting of alkyl, cyano, halo, and haloalkyl;
n is 0, 1, 2, or 3;
R^{a} is selected from the group consisting of C₆₋₁₀ cycloalkyl, substituted C₆₋₁₀ cycloalkyl, and

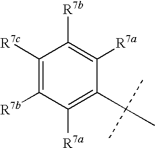

wherein each R^{7a} is independently hydrogen or halo;
each R^{7b} and R^{7c} are independently selected from the group consisting of hydrogen, halo, alkyl, acyl, acyloxy, carboxyl ester, acylamino, alkylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, alkylsulfonyl and haloalkylsulfonyl; provided that if one of R^{7b} is hydrogen and the other R^{7b} is halo, then R^{7c} and is not the same halo;
with the proviso that when m is 1, and X, Y and Z with the phenyl ring define an indole or benzimidazole, then at least one of X, Y, or Z is selected from —NR⁶— and —CR³═, where R⁶ or R³ is not hydrogen; and
with the proviso that Formula (II) is not:

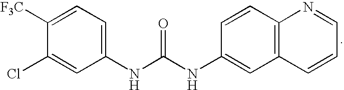

In another embodiment, provided are compounds of Formula (III), or a pharmaceutically acceptable salt thereof:

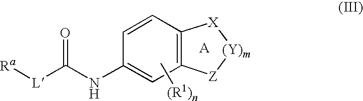

wherein:
A is an optionally substituted cycloalkyl, optionally substituted heterocyclic or optionally substituted heteroaryl ring fused with the benzene ring;
each of X, Y and Z are independently selected from the group consisting of —CR³═, —C(R⁴)(R⁵)—, —C(═O)—, —C(═S)—, —N═, —NR⁶—, —O—, —S—, —SO—, and —SO₂—, provided that at least one of X, Y and Z is selected from the group consisting of —C(═O)—, —C(═S)—, —N═, —NR⁶—, —O—, —S—, —SO—, and —SO₂—; and provided that X and Z are not both —C(═O)— and that A is not substituted with four fluoro;
   wherein
   each R³, R⁴ and R⁵ is independently selected from the group consisting of R⁶, halo, alkoxy, substituted alkoxy, acyloxy, carboxyl ester, acylamino, alkylamino, aminocarbonylamino, aminocarbonyloxy, aminosulfonylamino, (substituted sulfonyl)amino, (substituted sulfonyl)aminocarbonyl, (carboxyl ester)amino;
   R⁶ is independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclyl, substituted heterocyclyl, haloalkyl, acyl, aminosulfonyl, and aminocarbonyl;
m is 1, 2 or 3;
L′ is a covalent bond or —CR′R″— where R′ and R″ are independently H or alkyl or R′ and R″ together form a C₃-C₆ cycloalkyl ring;
each R¹ is independently selected from the group consisting of alkyl, cyano, halo, and haloalkyl;
n is 0, 1, 2, or 3; and
R^{a} is selected from the group consisting of C₆₋₁₀ cycloalkyl, substituted C₆₋₁₀ cycloalkyl, and

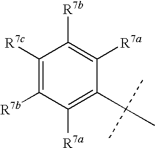

wherein each R^{7a} is independently hydrogen or halo;
each R^{7b} and R^{7c} are independently selected from the group consisting of hydrogen, halo, alkyl, acyl, acyloxy, carboxyl ester, acylamino, alkylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, alkylsulfonyl and haloalkylsulfonyl; provided that if one of R^{7b} is hydrogen and the other R^{7b} is halo, then R^{7c} and is not the same halo.

In other embodiments, provided is a compound of Formula (IVa), (IVb) or (IVc), or a pharmaceutically acceptable salt thereof:

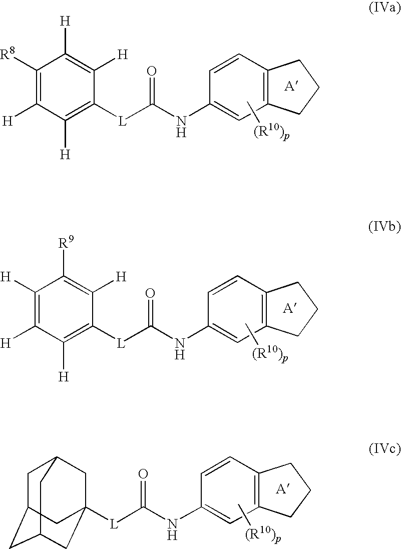

wherein
A′ is a five or six membered optionally substituted heterocyclic or optionally substituted heteroaryl ring having at least one ring nitrogen atom and is fused with the phenyl ring;
each R¹⁰ is independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclyl, substituted heterocyclyl, haloalkyl, acyl, aminosulfonyl, and aminocarbonyl, halo, alkoxy, substituted alkoxy, acyloxy, carboxyl ester, acylamino, alkylamino, aminocarbonylamino, aminocarbonyloxy, aminosulfonylamino, (substituted sulfonyl)amino, (substituted sulfonyl)aminocarbonyl, (carboxyl ester)amino;
p is 0, 1, 2, or 3;
R⁸ and R⁹ are selected from the group consisting of halo, haloalkyl, haloalkoxy, alkylthio, haloalkylthio, cyano, alkylsulfonyl, and haloalkylsulfonyl; and
L is as previously defined.

In another embodiment, provided are compounds of Table 1 or a pharmaceutically acceptable salt thereof.

In accordance with another aspect of the invention, provided is a method for treating a soluble expoxide hydrolase mediated disease, said method comprising administering to a patient a pharmaceutical composition comprising a pharmaceutically acceptable carrier and a therapeutically effective amount of a compound of the invention or a pharmaceutically acceptable salt thereof.

In accordance with yet another aspect of the invention, provided is a method for inhibiting a soluble expoxide hydrolase, said method comprising contacting the soluble epoxide hydrolase with an effective amount of a compound of the invention or a pharmaceutically acceptable salt thereof.

### DETAILED DESCRIPTION OF THE INVENTION

### Definitions

As used herein, the following definitions shall apply unless otherwise indicated.

“cis-Epoxyeicosatrienoic acids” (“EETs”) are biomediators synthesized by cytochrome P450 epoxygenases.

“Epoxide hydrolases” (“EH;” EC 3.3.2.3) are enzymes in the alpha/beta hydrolase fold family that add water to 3 membered cyclic ethers termed epoxides.

“Soluble epoxide hydrolase” (“sEH”) is an enzyme which in endothelial, smooth muscle and other cell types converts EETs to dihydroxy derivatives called dihydroxyeicosatrienoic acids (“DHETs”). The cloning and sequence of the murine sEH is set forth in Grant et al., J. Biol. Chem. 268(23):17628-17633 (1993). The cloning, sequence, and accession numbers of the human sEH sequence are set forth in Beetham et al., Arch. Biochem. Biophys. 305(1):197-201 (1993). The amino acid sequence of human sEH is also set forth as SEQ ID NO:2 of U.S. Pat. No. 5,445,956; the nucleic acid sequence encoding the human sEH is set forth as nucleotides 42-1703 of SEQ ID NO:1 of that patent. The evolution and nomenclature of the gene is discussed in Beetham et al., DNA Cell Biol. 14(1):61-71 (1995). Soluble epoxide hydrolase represents a single highly conserved gene product with over 90% homology between rodent and human (Arand et al., FEBS Lett., 338:251-256 (1994)).

“Chronic Obstructive Pulmonary Disease” or “COPD” is also sometimes known as “chronic obstructive airway disease”, “chronic obstructive lung disease”, and “chronic airways disease.” COPD is generally defined as a disorder characterized by reduced maximal expiratory flow and slow forced emptying of the lungs. COPD is considered to encompass two related conditions, emphysema and chronic bronchitis. COPD can be diagnosed by the general practitioner using art recognized techniques, such as the patient's forced vital capacity (“FVC”), the maximum volume of air that can be forcibly expelled after a maximal inhalation. In the offices of general practitioners, the FVC is typically approximated by a 6 second maximal exhalation through a spirometer. The definition, diagnosis and treatment of COPD, emphysema, and chronic bronchitis are well known in the art and discussed in detail by, for example, Honig and Ingram, in Harrison's Principles of Internal Medicine, (Fauci et al., Eds), 14th Ed., 1998, McGraw-Hill, New York, pp. 1451-1460 (hereafter, “Harrison's Principles of Internal Medicine”). As the names imply, “obstructive pulmonary disease” and “obstructive lung disease” refer to obstructive diseases, as opposed to restrictive diseases. These diseases particularly include COPD, bronchial asthma, and small airway disease.

“Emphysema” is a disease of the lungs characterized by permanent destructive enlargement of the airspaces distal to the terminal bronchioles without obvious fibrosis.

“Chronic bronchitis” is a disease of the lungs characterized by chronic bronchial secretions which last for most days of a month, for three months, a year, for two years, etc.

“Small airway disease” refers to diseases where airflow obstruction is due, solely or predominantly to involvement of the small airways. These are defined as airways less than 2 mm in diameter and correspond to small cartilaginous bronchi, terminal bronchioles, and respiratory bronchioles. Small airway disease (SAD) represents luminal obstruction by inflammatory and fibrotic changes that increase airway resistance. The obstruction may be transient or permanent.

“Interstitial lung diseases (ILDs)” are restrictive lung diseases involving the alveolar walls, perialveolar tissues, and contiguous supporting structures. As discussed on the website of the American Lung Association, the tissue between the air sacs of the lung is the interstitium, and this is the tissue affected by fibrosis in the disease. Persons with such restrictive lung disease have difficulty breathing in because of the stiffness of the lung tissue but, in contrast to persons with obstructive lung disease, have no difficulty breathing out. The definition, diagnosis and treatment of interstitial lung diseases are well known in the art and discussed in detail by, for example, Reynolds, H. Y., in Harrison's Principles of Internal Medicine, supra, at pp. 1460-1466. Reynolds notes that, while ILDs have various initiating events, the immunopathological responses of lung tissue are limited and the ILDs therefore have common features.

“Idiopathic pulmonary fibrosis,” or “IPF,” is considered the prototype ILD. Although it is idiopathic in that the cause is not known, Reynolds, supra, notes that the term refers to a well defined clinical entity.

“Bronchoalveolar lavage,” or “BAL,” is a test which permits removal and examination of cells from the lower respiratory tract and is used in humans as a diagnostic procedure for pulmonary disorders such as IPF. In human patients, it is usually performed during bronchoscopy.

“Diabetic neuropathy” refers to acute and chronic peripheral nerve dysfunction resulting from diabetes.

“Diabetic nephropathy” refers to renal diseases resulting from diabetes.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Alkenyl” refers to straight or branched hydrocarbyl groups having from 2 to 6 carbon atoms and preferably 2 to 4 carbon atoms and having at least 1 and preferably from 1 to 2 sites of vinyl (>C═C<) unsaturation. Such groups are exemplified, for example, by vinyl, allyl, and but-3-en-1-yl. Included within this term are the cis and trans isomers or mixtures of these isomers.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic (—C≡C—) unsaturation. Examples of such alkynyl groups include acetylenyl (—C≡CH), and propargyl (—CH₂C≡CH).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to a vinyl (unsaturated) carbon atom.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to an acetylenic carbon atom.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Acylamino” refers to the groups —NR¹⁷C(O)alkyl, —NR¹⁷C(O)substituted alkyl, —NR¹⁷C(O)cycloalkyl, —NR¹⁷C(O)substituted cycloalkyl, —NR¹⁷C(O)cycloalkenyl, —NR¹⁷C(O)substituted cycloalkenyl, —NR¹⁷C(O)alkenyl, —NR¹⁷C(O)substituted alkenyl, —NR¹⁷C(O)alkynyl, —NR¹⁷C(O)substituted alkynyl, —NR¹⁷C(O)aryl, —NR¹⁷C(O)substituted aryl, —NR¹⁷C(O)heteroaryl, —NR¹⁷C(O)substituted heteroaryl, —NR¹⁷C(O)heterocyclic, and —NR¹⁷C(O)substituted heterocyclic wherein R¹⁷ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group —NR¹⁸R¹⁹ where R¹⁸ and R¹⁹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cycloalkenyl, —SO₂-aryl, —SO₂-substituted aryl, —SO₂-heteroaryl, —SO₂-substituted heteroaryl, —SO₂-heterocyclic, and —SO₂-substituted heterocyclic and wherein R¹⁸ and R¹⁹ are optionally joined, together with the nitrogen bound thereto to form a heterocyclic or substituted heterocyclic group, provided that R¹⁸ and R¹⁹ are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. When R¹⁸ is hydrogen and R¹⁹ is alkyl, the substituted amino group is sometimes referred to herein as alkylamino. When R¹⁸ and R¹⁹ are alkyl, the substituted amino group is sometimes referred to herein as dialkylamino. When referring to a monosubstituted amino, it is meant that either R¹⁸ or R¹⁹ is hydrogen but not both. When referring to a disubstituted amino, it is meant that neither R¹⁸ nor R¹⁹ are hydrogen.

“Aminocarbonyl” refers to the group —C(O)NR²⁰R²¹ where R²⁰ and R²¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²⁰ and R²¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonyl” refers to the group —C(S)NR²⁰R²¹ where R²⁰ and R²¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²⁰ and R²¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR¹⁷C(O)NR²⁰R²¹ where R¹⁷ is hydrogen or alkyl and R²⁰ and R²¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²⁰ and R²¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonylamino” refers to the group —NR¹⁷C(S)NR²⁰R²¹ where R¹⁷ is hydrogen or alkyl and R²⁰ and R²¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyloxy” refers to the group —O—C(O)NR²⁰R²¹ where R²⁰ and R²¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²⁰ and R²¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyl” refers to the group —SO₂NR²⁰R²¹ where R²⁰ and R²¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²⁰ and R²¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyloxy” refers to the group —O—SO₂NR²⁰R²¹ where R²⁰ and R²¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²⁰ and R²¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonylamino” refers to the group —NR¹⁷—SO₂NR²⁰R²¹ where R¹⁷ is hydrogen or alkyl and R²⁰ and R²¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²⁰ and R²¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amidino” refers to the group —C(═NR²²)NR²⁰R²¹ where R²⁰, R²¹, and R²² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²⁰ and R²¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Carbonyl” refers to the divalent group —C(O)— which is equivalent to —C(═O)—.

“Carboxy” or “carboxyl” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)amino” refers to the group —NR¹⁷—C(O)O-alkyl, —NR¹⁷—C(O)O-substituted alkyl, —NR¹⁷—C(O)O-alkenyl, —NR¹⁷—C(O)O-substituted alkenyl, —NR¹⁷—C(O)O-alkynyl, —NR¹⁷—C(O)O-substituted alkynyl, —NR¹⁷—C(O)O-aryl, —NR¹⁷—C(O)O-substituted aryl, —NR¹⁷—C(O)O-cycloalkyl, —NR¹⁷—C(O)O-substituted cycloalkyl, —NR¹⁷—C(O)O-cycloalkenyl, —NR¹⁷—C(O)O-substituted cycloalkenyl, —NR¹⁷—C(O)O-heteroaryl, —NR¹⁷—C(O)O-substituted heteroaryl, —NR¹⁷—C(O)O-heterocyclic, and —NR¹⁷—C(O)O-substituted heterocyclic wherein R¹⁷ is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, substituted —O—C(O)O-alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” refers to the group —CN.

“Cycloalkyl” refers to cyclic alkyl groups of from 3 to 10 carbon atoms having single or multiple cyclic rings including fused, bridged, and spiro ring systems. One or more of the rings can be aryl, heteroaryl, or heterocyclic provided that the point of attachment is through the non-aromatic, non-heterocyclic ring carbocyclic ring. Examples of suitable cycloalkyl groups include, for instance, adamantyl, cyclopropyl, cyclobutyl, cyclopentyl, and cyclooctyl. Other examples of cycloalkyl groups include bicycle[2,2,2,]octanyl, norbornyl, and spirobicyclo groups such as spiro[4.5]dec-8-yl:

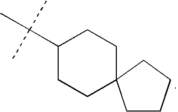

“Cycloalkenyl” refers to non-aromatic cyclic alkyl groups of from 3 to 10 carbon atoms having single or multiple cyclic rings and having at least one >C═C< ring unsaturation and preferably from 1 to 2 sites of >C═C< ring unsaturation.

“Substituted cycloalkyl” and “substituted cycloalkenyl” refers to a cycloalkyl or cycloalkenyl group having from 1 to 5 or preferably 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Cycloalkyloxy” refers to —O-cycloalkyl.

“Substituted cycloalkyloxy refers to —O-(substituted cycloalkyl).

“Cycloalkylthio” refers to —S-cycloalkyl.

“Substituted cycloalkylthio” refers to —S-(substituted cycloalkyl).

“Cycloalkenyloxy” refers to —O-cycloalkenyl.

“Substituted cycloalkenyloxy refers to —O-(substituted cycloalkenyl).

“Cycloalkenylthio” refers to —S-cycloalkenyl.

“Substituted cycloalkenylthio” refers to —S-(substituted cycloalkenyl).

“Guanidino” refers to the group —NHC(═NH)NH₂.

“Substituted guanidino” refers to —NR²³C(═NR²³)N(R²³)₂ where each R²³ is independently selected from the group consisting of hydrogen, alkyl, substituted alkyl, aryl, substituted aryl, heteroaryl, substituted heteroaryl, heterocyclic, and substituted heterocyclic and two R²³ groups attached to a common guanidino nitrogen atom are optionally joined together with the nitrogen bound thereto to form a heterocyclic or substituted heterocyclic group, provided that at least one R²³ is not hydrogen, and wherein said substituents are as defined herein.

“Halo” or “halogen” refers to fluoro, chloro, bromo and iodo and preferably is fluoro or chloro.

“Haloalkyl” refers to alkyl groups substituted with 1 to 5, 1 to 3, or 1 to 2 halo groups, wherein alkyl and halo are as defined herein.

“Haloalkoxy” refers to alkoxy groups substituted with 1 to 5, 1 to 3, or 1 to 2 halo groups, wherein alkoxy and halo are as defined herein.

“Haloalkylthio” refers to alkylthio groups substituted with 1 to 5, 1 to 3, or 1 to 2 halo groups, wherein alkylthio and halo are as defined herein.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, and/or sulfonyl moieties. Preferred heteroaryls include pyridinyl, pyrrolyl, indolyl, thiophenyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heteroaryloxy” refers to —O-heteroaryl.

“Substituted heteroaryloxy refers to the group —O-(substituted heteroaryl).

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” refers to a saturated or partially saturated, but not aromatic, group having from 1 to 10 ring carbon atoms and from 1 to 4 ring heteroatoms selected from the group consisting of nitrogen, sulfur, or oxygen. Heterocycle encompasses single ring or multiple condensed rings, including fused bridged and spiro ring systems. In fused ring systems, one or more the rings can be cycloalkyl, aryl, or heteroaryl provided that the point of attachment is through the non-aromatic heterocyclic ring. In one embodiment, the nitrogen and/or sulfur atom(s) of the heterocyclic group are optionally oxidized to provide for the N-oxide, sulfinyl, and/or sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocycyl.

“Substituted heterocyclyloxy refers to the group —O-(substituted heterocycyl).

“Heterocyclylthio” refers to the group —S-heterocycyl.

“Substituted heterocyclylthio” refers to the group —S-(substituted heterocycyl).

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O) or (—O⁻).

“Spiro ring systems” refers to bicyclic ring systems that have a single ring carbon atom common to both rings.

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cycloalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Substituted sulfonyl includes groups such as methyl-SO₂—, phenyl-SO₂—, and 4-methylphenyl-SO₂—. The term “alkylsulfonyl” refers to —SO₂-alkyl. The term “haloalkylsulfonyl” refers to —SO₂-haloalkyl where haloalkyl is defined herein. The term “(substituted sulfonyl)amino” refers to —NH(substituted sulfonyl), and the term “(substituted sulfonyl)aminocarbonyl” refers to —C(O)NH(substituted sulfonyl), wherein substituted sulfonyl is as defined herein.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cylcoalkyl, —OSO₂-cycloalkenyl, —OSO₂-substituted cycloalkenyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thiol” refers to the group —SH.

“Thiocarbonyl” refers to the divalent group —C(S)— which is equivalent to —C(═S)—.

“Thione” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein.

“Compound” or “compounds” as used herein is meant to include the stereoiosmers and tautomers of the indicated formulas.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Patient” refers to mammals and includes humans and non-human mammals.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate.

“Treating” or “treatment” of a disease in a patient refers to (1) preventing the disease from occurring in a patient that is predisposed or does not yet display symptoms of the disease; (2) inhibiting the disease or arresting its development; or (3) ameliorating or causing regression of the disease.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—; “alkylsulfonyl” refers to alkyl-SO₂—; and “haloalkylthio” refers to haloalkyl-S—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group etc) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

Accordingly, the present invention provides a compound of Formula (I) or a pharmaceutically acceptable salt thereof:

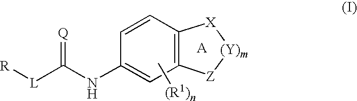

wherein:
A is an optionally substituted cycloalkyl, optionally substituted heterocyclic or optionally substituted heteroaryl ring fused with the benzene ring;
each of X, Y and Z are independently selected from the group consisting of —CR³═, —C(R⁴)(R⁵)—, —C(═O)—, —C(═S)—, —N═, —NR⁶—, —O—, —S—, —SO—, and —SO₂—, provided that at least one of X, Y and Z is selected from the group consisting of —C(═O)—, —C(═S)—, —N═, —NR⁶—, —O—, —S—, —SO—, and —SO₂—; and provided that X and Z are not both C(═O) and that A is not substituted with four fluoro;
   wherein
   each R³, R⁴ and R⁵ is independently selected from the group consisting of R⁶, halo, alkoxy, substituted alkoxy, acyloxy, carboxyl ester, acylamino, alkylamino, aminocarbonylamino, aminocarbonyloxy, aminosulfonylamino, (substituted sulfonyl)amino, (substituted sulfonyl)aminocarbonyl, (carboxyl ester)amino;
   R⁶ is independently selected from the group consisting of hydrogen, alkyl, substituted alkyl, alkenyl, substituted alkenyl, alkynyl, substituted alkynyl, heteroaryl, heterocyclyl, haloalkyl, acyl, aminosulfonyl, and aminocarbonyl;
m is 1, 2 or 3;
Q is O or S;
L is a covalent bond, —NH—, or —CR′R″— where R′ and R″ are independently H or alkyl or R′ and R″ together form a C₃-C₆ cycloalkyl ring;
each R¹ is independently selected from the group consisting of alkyl, cyano, halo, and haloalkyl;
n is 0, 1, 2, or 3;
R is selected from the group consisting of C₆₋₁₀ cycloalkyl, substituted C₆₋₁₀ cycloalkyl and

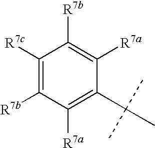

wherein each R^{7a} is independently hydrogen or halo;
each of R^{7b} and R^{7c} is independently selected from the group consisting of hydrogen, halo, alkyl, acyl, acyloxy, carboxyl ester, acylamino, alkylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, alkylsulfonyl and haloalkylsulfonyl; or R^{7c} and one of R^{7b} together form a heterocycloalkyl ring; provided that if one of R^{7b} is hydrogen and the other R^{7b} is halo, then R^{7c} and is not the same halo;
with the proviso that when m is 1, and X, Y and Z with the phenyl ring define an indole or benzimidazole, then at least one of X, Y, or Z is selected from —NR⁶— and —CR³═, where R⁶ or R³ is not hydrogen; and
with the proviso that Formula (I) is not:

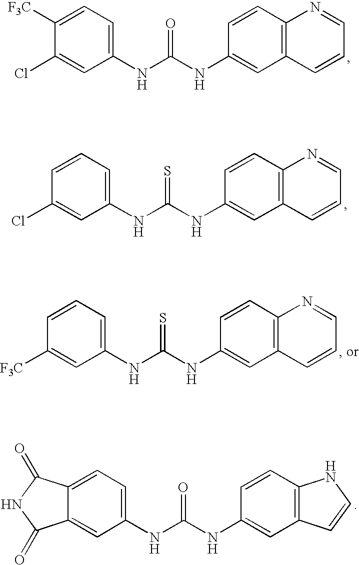

In another embodiment, provided is a compound of Formula (II) or a pharmaceutically acceptable salt thereof:

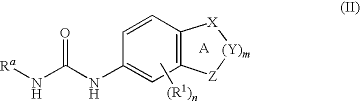

wherein:
A is an optionally substituted cycloalkyl, optionally substituted heterocyclic or optionally substituted heteroaryl ring fused with the benzene ring;
each of X, Y and Z are independently selected from the group consisting of —CR³═, —C(R⁴)(R⁵)—, —C(═O)—, —C(═S)—, ═N—, —NR⁶—, —O—, —S—, —SO—, and —SO₂—, provided that at least one of X, Y and Z is selected from the group consisting of —C(═O)—, —C(═S)—, —N═, —NR⁶—, —O—, —S—, —SO—, and —SO₂—; and provided that X and Z are not both —C(═O)— and that A is not substituted with four fluoro;
   wherein
   each R³, R⁴ and R⁵ is independently selected from the group consisting of R⁶, halo, alkoxy, substituted alkoxy, acyloxy, carboxyl ester, acylamino, alkylamino, aminocarbonylamino, aminocarbonyloxy, aminosulfonylamino, (substituted sulfonyl)amino, (substituted sulfonyl)aminocarbonyl, (carboxyl ester)amino;
   R⁶ is independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclyl, substituted heterocyclyl, haloalkyl, acyl, aminosulfonyl, and aminocarbonyl;
m is 1, 2 or 3;
each R¹ is independently selected from the group consisting of alkyl, cyano, halo, and haloalkyl;
n is 0, 1, 2, or 3;
R^{a} is selected from the group consisting of C₆₋₁₀ cycloalkyl, substituted C₆₋₁₀ cycloalkyl and

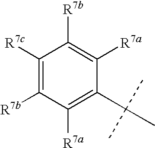

wherein each R^{7a} is independently hydrogen or halo;
each R^{7b} and R^{7c} are independently selected from the group consisting of hydrogen, halo, alkyl, acyl, acyloxy, carboxyl ester, acylamino, alkylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, alkylsulfonyl and haloalkylsulfonyl; provided that if one of R^{7b} is hydrogen and the other R^{7b} is halo, then R^{7c} and is not the same halo;
with the proviso that when m is 1, and X, Y and Z with the phenyl ring define an indole or benzimidazole, then at least one of X, Y, or Z is selected from —NR⁶— and —CR³═, where R⁶ or R³ is not hydrogen; and
with the proviso that Formula (II) is not:

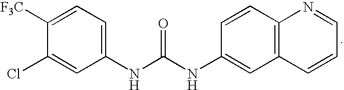

Various embodiments relating to the compounds of Formula (I) or (II) or pharmaceutically acceptable salts are listed below. These embodiments can be combined with each other or with any other embodiments described in this application. In some aspects, provided are compounds of Formula (I) or (II) having one or more of the following features.

In some embodiments of Formula (I), L is a covalent bond. In some embodiments L is —NH—. In some embodiments, L is —CR′R″— where R′ and R″ are independently H or alkyl or R′ and R″ together form a C₃-C₆ cycloalkyl ring. In some embodiments, L is —CH₂—.

In some embodiments of Formula (I) or (II),

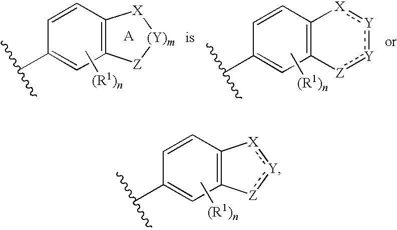

wherein
each X, Y, and Z is independently selected from the group consisting of —CR³—, —N═, —C(R⁴)(R⁵)—, —C(═O)—, —C(═S)—, —NR⁶—, —O—, —S—, —SO—, and —SO₂—; and
each [symbol] independently represents a sing bond or a double bond, provided that two double bonds are not adjacent to each other.

In some embodiments of Formula (I) or (II), at least one of X, Y and Z is selected from the group consisting of —N═, —NR⁶— —O—, —S—, —SO—, and —SO₂—. In some embodiments, at least one of X, Y and Z is —N═ or —NR⁶—.

In some embodiments of Formula (I) or (II),

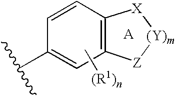

is selected from the group consisting of:

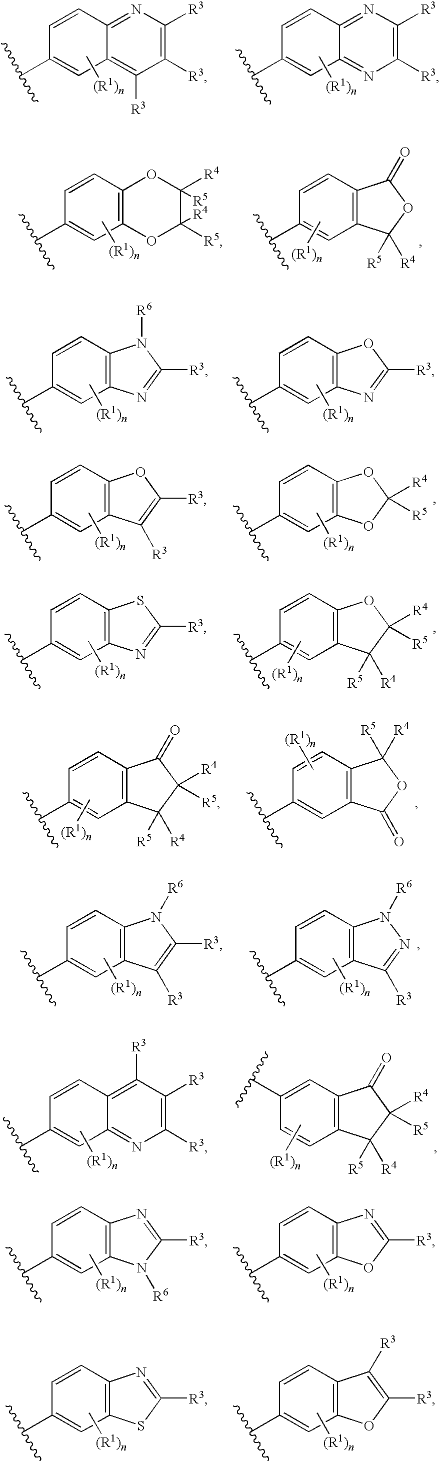

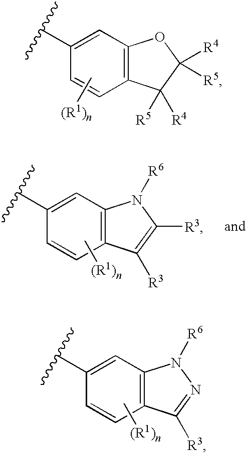

wherein each of R¹, R³, R⁴, R⁵ and R⁶ is as defined above.

In some embodiments,

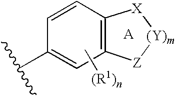

is selected from the group consisting of:

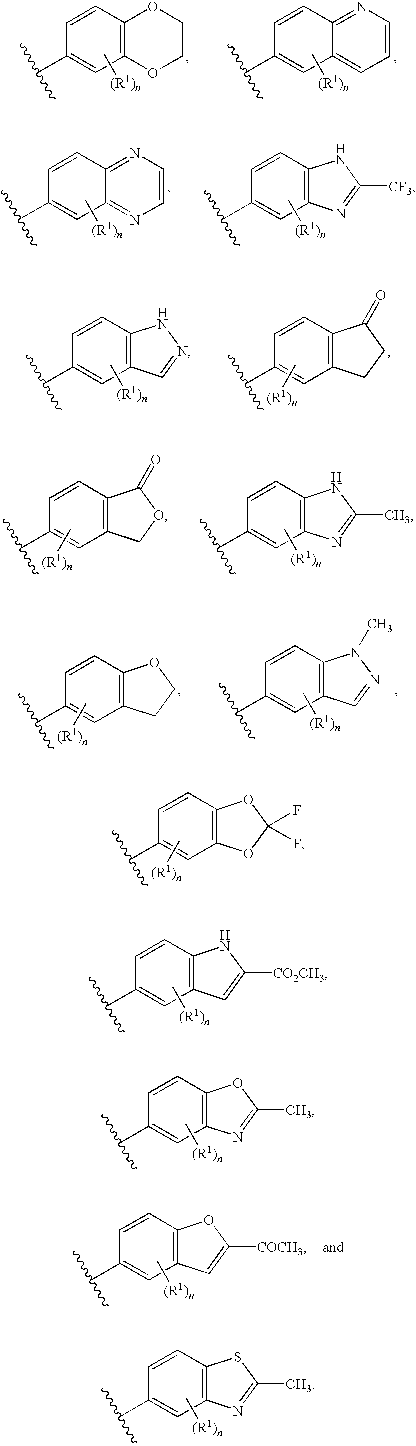

In some embodiments of Formula (I), Q is O.

In some embodiments of Formula (I), Q is S.

In some embodiments of Formula (I) or (II), n is 0.

In some embodiments n is 1 and R¹ is halo. In some embodiments, R¹ is fluoro.

In some embodiments of Formula (I) or (II) R or R^{a} is C₆₋₁₀ cycloalkyl or substituted C₆₋₁₀ cycloalkyl.

In some embodiments, R or R^{a} is selected from the group consisting of

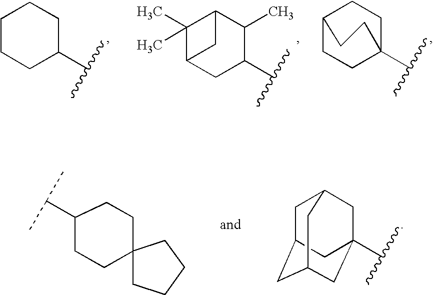

In some embodiments, R or R^{a} is adamantyl.

In some embodiments, R or R^{a} is

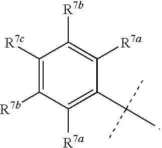

In some embodiments both R^{7a} are hydrogen.

In some embodiments at least one of R^{7a} is fluoro or chloro. In some embodiments one of R^{7a} is fluoro, and the other of R^{7a} is hydrogen.

In some embodiments each R^{7b} and R^{7c} are independently selected from the group consisting of hydrogen, halo, alkyl, haloalkyl, haloalkoxy, alkylamino, heterocycloalkyloxy, alkylthio, haloalkylthio, cyano, alkylsulfonyl, and haloalkylsulfonyl.

In some embodiments at least one of R^{7b} and R^{7c} is selected from the group consisting of halo, alkyl, haloalkyl, haloalkoxy, alkylamino, heterocycloalkyloxy, alkylthio, haloalkylthio, cyano, alkylsulfonyl, and haloalkylsulfonyl.

In some embodiments one of R^{7b} and R^{7c} is selected from the group consisting of halo, alkyl, haloalkyl, haloalkoxy, alkylamino, heterocycloalkyloxy, alkylthio, haloalkylthio, cyano, alkylsulfonyl, and haloalkylsulfonyl, and the remainder of R^{7b} and R^{7c} are hydrogen.

In some embodiments at least one of R^{7b} and R^{7c} is selected from the group consisting of halo, trifluoromethyl, trifluoromethoxy, alkylsulfonyl, and haloalkylsulfonyl.

In some embodiments R^{7c} is selected from the group consisting of chloro, fluoro, trifluoromethyl, and trifluoromethoxy. In some embodiments, R^{7b} and R^{7a} are hydrogen.

In some embodiments, R or R^{a} is selected from the group consisting of 3-trifluoromethylphenyl, 4-trifluoromethylphenyl, 3-trifluoromethoxyphenyl, 4-trifluoromethoxyphenyl, 4-fluorophenyl, and 4-chlorophenyl.

In other embodiments, provided is a compound of Formula (III) or a pharmaceutically acceptable salt thereof:

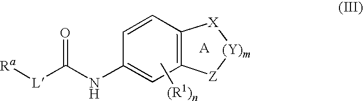

A is an optionally substituted cycloalkyl, optionally substituted heterocyclic or optionally substituted heteroaryl ring fused with the benzene ring;
each of X, Y and Z are independently selected from the group consisting of —CR³═, —C(R⁴)(R⁵)—, —C(═O)—, —C(═S)—, —N═, —NR⁶—, —O—, —S—, —SO—, and —SO₂—, provided that at least one of X, Y and Z is selected from the group consisting of —C(═O)—, —C(═S)—, —N═, —NR⁶—, —O—, —S—, —SO—, and —SO₂—; and provided that X and Z are not both —C(═O)— and that A is not substituted with four fluoro;
   wherein
   each R³, R⁴ and R⁵ is independently selected from the group consisting of R⁶, halo, alkoxy, substituted alkoxy, acyloxy, carboxyl ester, acylamino, alkylamino, aminocarbonylamino, aminocarbonyloxy, aminosulfonylamino, (substituted sulfonyl)amino, (substituted sulfonyl)aminocarbonyl, (carboxyl ester)amino;
   R⁶ is independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclyl, substituted heterocyclyl, haloalkyl, acyl, aminosulfonyl, and aminocarbonyl;
m is 1, 2 or 3;
L′ a covalent bond or —CR′R″— where R′ and R″ are independently H or alkyl or R′ and R″ together form a C₃-C₆ cycloalkyl ring;
each R¹ is independently selected from the group consisting of alkyl, cyano, halo, and haloalkyl;
n is 0, 1, 2, or 3; and
R^{a} is selected from the group consisting of C₆₋₁₀ cycloalkyl, substituted C₆₋₁₀ cycloalkyl, and

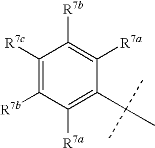

wherein each R^{7a} is independently hydrogen or halo;
each R^{7b} and R^{7c} are independently selected from the group consisting of hydrogen, halo, alkyl, acyl, acyloxy, carboxyl ester, acylamino, alkylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, alkylsulfonyl and haloalkylsulfonyl; provided that if one of R^{7b} is hydrogen and the other R^{7b} is halo, then R^{7c} and is not the same halo.

Various embodiments relating to the compounds of Formula (III) or pharmaceutically acceptable salts thereof are listed below. These embodiments can be combined with each other or with any other embodiments described in this application. In some aspects, provided are compounds of Formula (III) having one or more of the following features.

In some embodiments of Formula (III) L′ is a covalent bond. In some embodiments, L′ is —CR′R″— where R′ and R″ are independently H or alkyl or R′ and R″ together form a C₃-C₆ cycloalkyl ring. In some embodiments, L′ is —CH₂—.

In some embodiments,

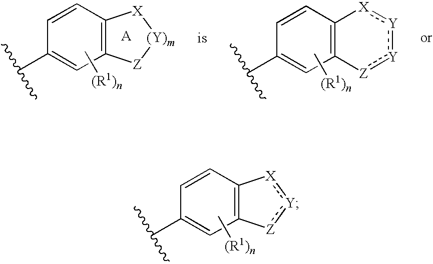

wherein
each X, Y, and Z is independently selected from the group consisting of —CR³═, —N═, —C(R⁴)(R⁵)—, —C(═O)—, —C(═S)—, —NR⁶—, —O—, —S—, —SO—, and —SO₂—; and
each [symbol] independently represents a sing bond or a double bond, provided that two double bonds are not adjacent to each other.

In some embodiments, at least one of X, Y and Z is selected from the group consisting of —N═, —NR⁶—, —O—, —S—, —SO—, and —SO₂—. In some embodiments at least one of X, Y and Z is —N═ or —NR⁶—.

In some embodiments,

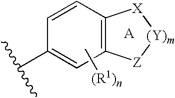

is selected from the group consisting of:

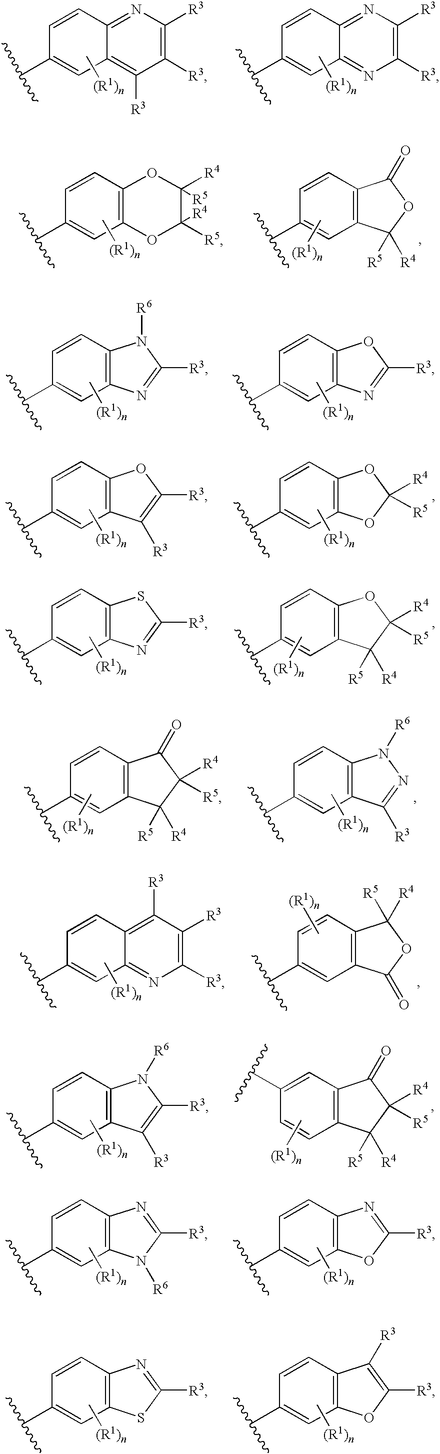

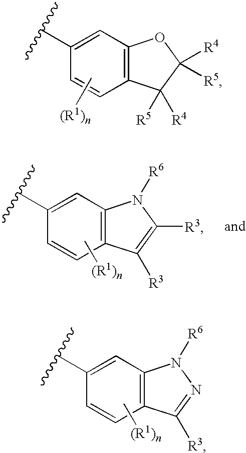

wherein each of R¹, R³, R⁴, R⁵ and R⁶ is as defined above.

In some embodiments, R³ is hydrogen, alkyl, acyl, carboxyl ester or haloalkyl.

In some embodiments, R⁴ and R⁵ on the same carbon are both hydrogen. In some embodiments, R⁴ and R⁵ on the same carbon are both halo.

In some embodiments, R⁶ is hydrogen or alkyl.

In some embodiments,

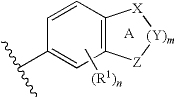

is selected from the group consisting of:

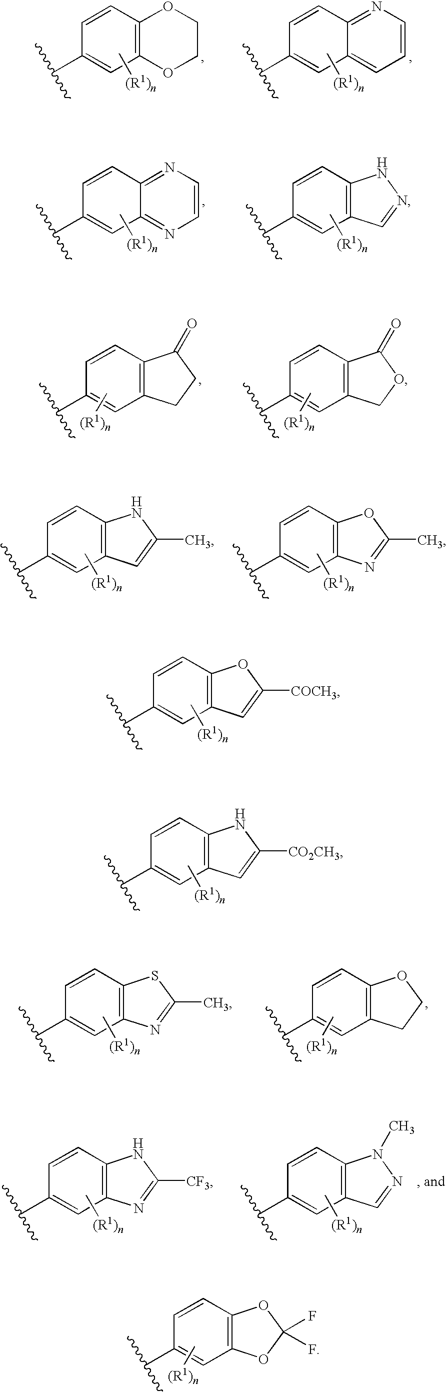

In some embodiments n is 0.

In some embodiments n is 1 and R¹ is halo. In some aspects, R¹ is fluoro.

In some embodiments, R^{a} is C₆₋₁₀ cycloalkyl or substituted C₆₋₁₀ cycloalkyl.

In some embodiments, R^{a} is selected from the group consisting of

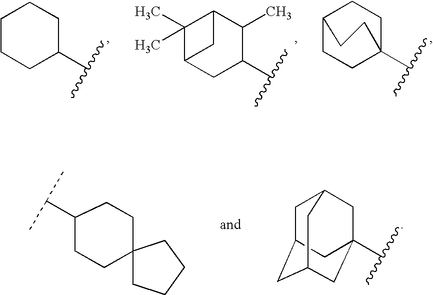

In some embodiments, R^{a} is adamantyl.

In some embodiments, R^{a} is

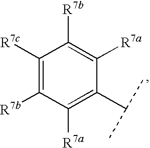

wherein each R^{7a} is independently hydrogen or halo;
each R^{7b} and R^{7c} are independently selected from the group consisting of hydrogen, halo, alkyl, acyl, acyloxy, carboxyl ester, acylamino, alkylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, alkylsulfonyl and haloalkylsulfonyl; provided that if one of R^{7b} is hydrogen and the other R^{7b} is halo, then R^{7c} and is not the same halo.

In some embodiments both R^{7a} are hydrogen.

In some embodiments at least one of R^{7a} is fluoro or chloro. In some embodiments one of R^{7a} is fluoro, and the other of R^{7a} is hydrogen.

In some embodiments each R^{7b} and R^{7c} are independently selected from the group consisting of hydrogen, halo, alkyl, haloalkyl, haloalkoxy, alkylamino, heterocycloalkyloxy, alkylthio, haloalkylthio, cyano, alkylsulfonyl, and haloalkylsulfonyl.

In some embodiments at least one of R^{7b} and R^{7c} is selected from the group consisting of halo, alkyl, haloalkyl, haloalkoxy, alkylamino, heterocycloalkyloxy, alkylthio, haloalkylthio, cyano, alkylsulfonyl, and haloalkylsulfonyl.

In some embodiments one of R^{7b} and R^{7c} is selected from the group consisting of halo, alkyl, haloalkyl, haloalkoxy, alkylamino, heterocycloalkyloxy, alkylthio, haloalkylthio, cyano, alkylsulfonyl, and haloalkylsulfonyl, and the remainder of R^{7b} and R^{7c} are hydrogen.

In some embodiments at least one of R^{7b} and R^{7c} is selected from the group consisting of halo, trifluoromethyl, trifluoromethoxy, alkylsulfonyl, and haloalkylsulfonyl.

In some embodiments R^{7c} is selected from the group consisting of chloro, fluoro, trifluoromethyl, and trifluoromethoxy. In some embodiments, all of R^{7a} and R^{7b} are hydrogen.

In some embodiments, R^{a} is selected from the group consisting of 4-fluorophenyl, 3-trifluoromethylphenyl, 4-trifluoromethylphenyl, 3-trifluoromethoxyphenyl, 4-trifluoromethoxyphenyl, and 4-chlorophenyl.

In other embodiments, provided is a compound of Formula (IVa), (IVb) or (IVc), or a pharmaceutically acceptable salt thereof:

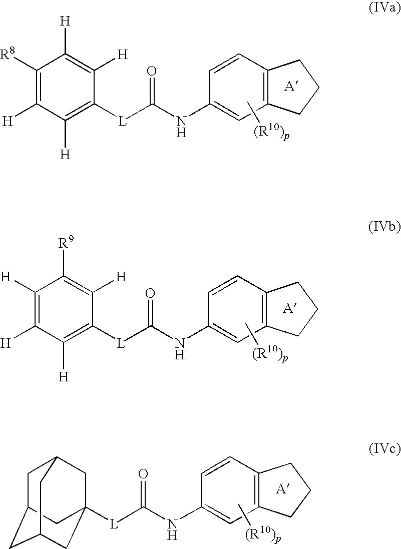

wherein
A′ is a five or six membered optionally substituted heterocyclic or optionally substituted heteroaryl ring having at least one ring nitrogen atom and is fused with the phenyl ring;
each R¹⁰ is independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclyl, substituted heterocyclyl, haloalkyl, acyl, aminosulfonyl, and aminocarbonyl, halo, alkoxy, substituted alkoxy, acyloxy, carboxyl ester, acylamino, alkylamino, aminocarbonylamino, aminocarbonyloxy, aminosulfonylamino, (substituted sulfonyl)amino, (substituted sulfonyl)aminocarbonyl, (carboxyl ester)amino;
p is 0, 1, 2, or 3;
R⁸ and R⁹ are selected from the group consisting of halo, haloalkyl, haloalkoxy, alkylthio, haloalkylthio, cyano, alkylsulfonyl, and haloalkylsulfonyl; and
L is a covalent bond, —NH—, or —CR′R″— where R′ and R″ are independently H or alkyl or R′ and R″ together form a C₃-C₆ cycloalkyl ring.

Various embodiments relating to the compounds or pharmaceutically acceptable salts of Formula (IVa), (IVb) and (IVc) are listed below. These embodiments can be combined with each other or with any other embodiments described in this application. In some aspects, provided are compounds of Formula (IVa), (IVb) or (IVc) having one or more of the following features.

In some embodiments of Formula (IVa), (IVb) or (IVc), L is —NH—. In some embodiments L is a covalent bond. In some embodiments, L is —CR′R″— where R′ and R″ are independently H or alkyl or R′ and R″ together form a C₃-C₆ cycloalkyl ring. In some embodiments, L is —CH₂—.

In some embodiments, A′ has at least one substituent selected from the group consisting of alkyl, acyl, carboxyl ester and haloalkyl.

In some embodiments p is 1 and R¹⁰ is halo. In some aspects, R¹⁰ is fluoro.

In some embodiments,

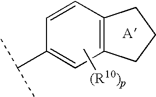

is selected from the group consisting of

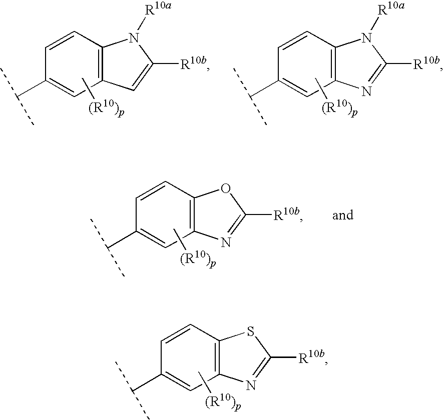

wherein
each R¹⁰ is independently selected from the group consisting of alkyl, halo, acyl, carboxyl ester and haloalkyl;
each R^{10a} is independently hydrogen or alkyl;
each R^{10b} is independently selected from the group consisting of hydrogen, alkyl, acyl, carboxyl ester and haloalkyl.

In some embodiments,

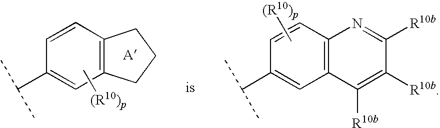

In some embodiments R⁸ and R⁹ are selected from the group consisting of halo, trifluoromethyl, trifluoromethoxy, alkylsulfonyl, and haloalkylsulfonyl. In some embodiments, R⁸ and R⁹ are selected from the group consisting of chloro, fluoro, trifluoromethoxy, and trifluoromethyl.

In some embodiments, provided is a compound or a pharmaceutically acceptable salt thereof selected from Table 1.

TABLE 1

| Compound | Structure | Name |
|---|---|---|
| 1 | 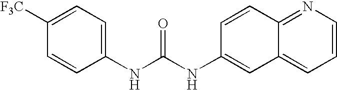 | 1-(quinolin-6-yl)-3-(4-(trifluoromethyl)phenyl)urea |
| 2 | 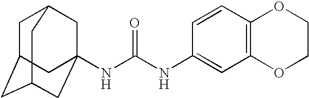 | 1-adamantan-1-yl-3-(2,3-dihydrobenzo[b][1,4]dioxin-6-yl)urea |
| 3 | 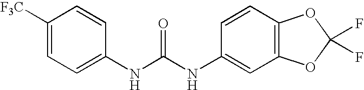 | 1-(2,2-difluorobenzol[d][1,3]dioxol-5-yl)-3-(4-(trifluoromethyl)phenyl)urea |
| 4 | 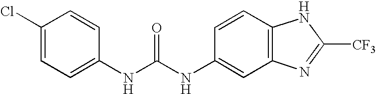 | 1-(4-chlorophenyl)-3-(2-(trifluoromethyl)-1H-benzo[d]imidazol-5-yl)urea |
| 5 | 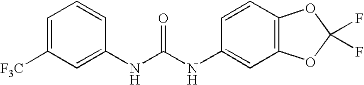 | 1-(2,2-difluorobenzo[d][1,3]dioxol-5-yl)-3-(3-(trifluoromethyl)phenyl)urea |
| 6 | 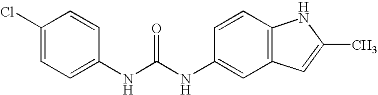 | 1-(4-chlorophenyl)-3-(2-methyl-1H-indol-5-yl)urea |
| 7 | 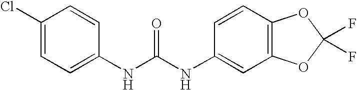 | 1-(4-chlorophenyl)-3-(2,2-difluorobenzo[d][1,3]dioxol-5-yl)urea |
| 8 | 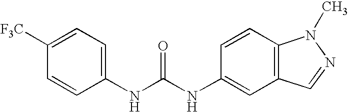 | 1-(1-methyl-1H-indazol-5-yl)-3-(4-(trifluoromethyl)phenyl)urea |
| 9 | 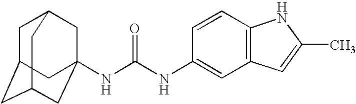 | 1-adamantan-1-yl-3-(2-methyl-1H-indol-5-yl)urea |
| 10 | 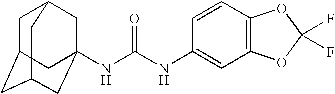 | 1-adamantan-1-yl-3-(2,2-difluorobenzo[d][1,3]dioxol-5-yl)urea |
| 11 | 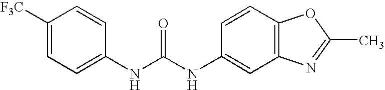 | 1-(2-methylbenzo[d]oxazol-5-yl)-3-(4-(trifluoromethyl)phenyl)urea |
| 12 | 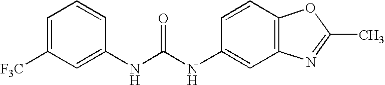 | 1-(2-methylbenzo[d]oxazol-5-yl)-3-(3-(trifluoromethyl)phenyl)urea |
| 13 | 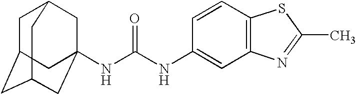 | 1-adamantan-1-yl-3-(2-methylbenzo[d]thiazol-5-yl)urea |
| 14 | 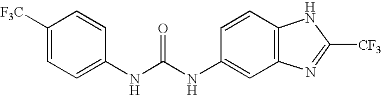 | 1-(2-(trifluoromethyl)-1H-benzo[d]imidazol-5-yl)-3-(4-(trifluoromethyl)phenyl)urea |
| 15 | 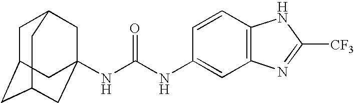 | 1-adamantan-1-yl-3-(2-(trifluoromethyl)-1H-benzo[d]imidazol-5-yl)urea |
| 16 | 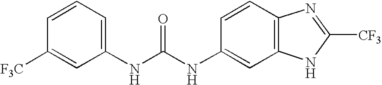 | 1-(2-(trifluoromethyl)-1H-benzo[d]imidazol-5-yl)-3-(3-(trifluoromethyl)phenyl)urea |
| 17 | 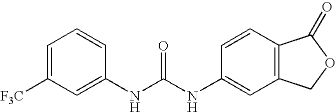 | 1-(1-oxo-1,3-dihydroisobenzofuran-5-yl)-3-(3-(trifluoromethyl)phenyl)urea |
| 18 | 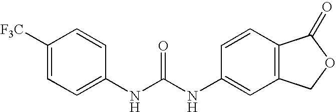 | 1-(1-oxo-1,3-dihydroisobenzofuran-5-yl)-3-(4-(trifluoromethyl)phenyl)urea |

In one embodiment, provided is a pharmaceutical composition comprising a pharmaceutically acceptable carrier and a therapeutically effective amount of a compound or pharmaceutically acceptable salt of any one of Formula (I)-(III) and (IVa)-(IVc) or of Table 1 for treating a soluble expoxide hydrolase mediated disease.

In another embodiment, provided is a method for treating a soluble expoxide hydrolase mediated disease, said method comprising administering to a patient a pharmaceutical composition comprising a pharmaceutically acceptable carrier and a therapeutically effective amount of a compound or pharmaceutically acceptable salt of any one of Formula (I)-(III) and (IVa)-(IVc) or of Table 1.

It has previously been shown that inhibitors of soluble epoxide hydrolase (“sEH”) can reduce hypertension (see, e.g., U.S. Pat. No. 6,351,506). Such inhibitors can be useful in controlling the blood pressure of persons with undesirably high blood pressure, including those who suffer from diabetes.

In preferred embodiments, compounds of the invention are administered to a subject in need of treatment for hypertension, specifically renal, hepatic, or pulmonary hypertension; inflammation, specifically renal inflammation, hepatic inflammation, vascular inflammation, and lung inflammation; adult respiratory distress syndrome; diabetic complications; end stage renal disease; Raynaud syndrome; and arthritis.

### Methods to Treat ARDS and SIRS

Adult respiratory distress syndrome (ARDS) is a pulmonary disease that has a mortality rate of 50% and results from lung lesions that are caused by a variety of conditions found in trauma patients and in severe burn victims. Ingram, R. H. Jr., “Adult Respiratory Distress Syndrome,” Harrison's Principals of Internal Medicine, 13, p. 1240, 1995. With the possible exception of glucocorticoids, there have not been therapeutic agents known to be effective in preventing or ameliorating the tissue injury, such as microvascular damage, associated with acute inflammation that occurs during the early development of ARDS.

ARDS, which is defined in part by the development of alveolar edema, represents a clinical manifestation of pulmonary disease resulting from both direct and indirect lung injury. While previous studies have detailed a seemingly unrelated variety of causative agents, the initial events underlying the pathophysiology of ARDS are not well understood. ARDS was originally viewed as a single organ failure, but is now considered a component of the multisystem organ failure syndrome (MOFS). Pharmacologic intervention or prevention of the inflammatory response is presently viewed as a more promising method of controlling the disease process than improved ventilatory support techniques. See, for example, Demling, Annu. Rev. Med., 46, pp. 193-203, 1995.

Another disease (or group of diseases) involving acute inflammation is the systematic inflammatory response syndrome, or SIRS, which is the designation recently established by a group of researchers to describe related conditions resulting from, for example, sepsis, pancreatitis, multiple trauma such as injury to the brain, and tissue injury, such as laceration of the musculature, brain surgery, hemorrhagic shock, and immune-mediated organ injuries (JAMA, 268(24):3452-3455 (1992)).

The ARDS ailments are seen in a variety of patients with severe burns or sepsis. Sepsis in turn is one of the SIRS symptoms. In ARDS, there is an acute inflammatory reaction with high numbers of neutrophils that migrate into the interstitium and alveoli. If this progresses there is increased inflammation, edema, cell proliferation, and the end result is impaired ability to extract oxygen. ARDS is thus a common complication in a wide variety of diseases and trauma. The only treatment is supportive. There are an estimated 150,000 cases per year and mortality ranges from 10% to 90%.

The exact cause of ARDS is not known. However it has been hypothesized that over-activation of neutrophils leads to the release of linoleic acid in high levels via phospholipase A₂ activity. Linoleic acid in turn is converted to 9,10-epoxy-12-octadecenoate enzymatically by neutrophil cytochrome P-450 epoxygenase and/or a burst of active oxygen. This lipid epoxide, or leukotoxin, is found in high levels in burned skin and in the serum and bronchial lavage of burn patients. Furthermore, when injected into rats, mice, dogs, and other mammals it causes ARDS. The mechanism of action is not known. However, the leukotoxin diol produced by the action of the soluble epoxide hydrolase appears to be a specific inducer of the mitochondrial inner membrane permeability transition (MPT). This induction by leukotoxin diol, the diagnostic release of cytochrome c, nuclear condensation, DNA laddering, and CPP32 activation leading to cell death were all inhibited by cyclosporin A, which is diagnostic for MPT induced cell death. Actions at the mitochondrial and cell level were consistent with this mechanism of action suggesting that the inhibitors of this invention could be used therapeutically with compounds which block MPT.

Thus in one embodiment provided is a method for treating ARDS. In another embodiment, provided is a method for treating SIRS.

### Methods for Inhibiting Progression of Kidney Deterioration (Nephropathy) and Reducing Blood Pressure:

In another aspect of the invention, the compounds of the invention can reduce damage to the kidney, and especially damage to kidneys from diabetes, as measured by albuminuria. The compounds of the invention can reduce kidney deterioration (nephropathy) from diabetes even in individuals who do not have high blood pressure. The conditions of therapeutic administration are as described above.

cis-Epoxyeicosantrienoic acids (“EETs”) can be used in conjunction with the compounds of the invention to further reduce kidney damage. EETs, which are epoxides of arachidonic acid, are known to be effectors of blood pressure, regulators of inflammation, and modulators of vascular permeability. Hydrolysis of the epoxides by sEH diminishes this activity. Inhibition of sEH raises the level of EETs since the rate at which the EETs are hydrolyzed into DHETs is reduced. Without wishing to be bound by theory, it is believed that raising the level of EETs interferes with damage to kidney cells by the microvasculature changes and other pathologic effects of diabetic hyperglycemia. Therefore, raising the EET level in the kidney is believed to protect the kidney from progression from microalbuminuria to end stage renal disease.

EETs are well known in the art. EETs useful in the methods of the present invention include 14,15-EET, 8,9-EET and 11,12-EET, and 5,6 EETs, in that order of preference. Preferably, the EETs are administered as the methyl ester, which is more stable. Persons of skill will recognize that the EETs are regioisomers, such as 8S,9R- and 14R,15S-EET. 8,9-EET, 11,12-EET, and 14R,15S-EET, are commercially available from, for example, Sigma-Aldrich (catalog nos. E5516, E5641, and E5766, respectively, Sigma-Aldrich Corp., St. Louis, Mo.).

EETs produced by the endothelium have anti-hypertensive properties and the EETs 11,12-EET and 14,15-EET may be endothelium-derived hyperpolarizing factors (EDHFs). Additionally, EETs such as 11,12-EET have profibrinolytic effects, anti-inflammatory actions and inhibit smooth muscle cell proliferation and migration. In the context of the present invention, these favorable properties are believed to protect the vasculature and organs during renal and cardiovascular disease states.

Inhibition of sEH activity can be effected by increasing the levels of EETs. This permits EETs to be used in conjunction with one or more sEH inhibitors to reduce nephropathy in the methods of the invention. It further permits EETs to be used in conjunction with one or more sEH inhibitors to reduce hypertension, or inflammation, or both. Thus, medicaments of EETs can be made which can be administered in conjunction with one or more sEH inhibitors, or a medicament containing one or more sEH inhibitors can optionally contain one or more EETs.

The EETs can be administered concurrently with the sEH inhibitor, or following administration of the sEH inhibitor. It is understood that, like all drugs, inhibitors have half lives defined by the rate at which they are metabolized by or excreted from the body, and that the inhibitor will have a period following administration during which it will be present in amounts sufficient to be effective. If EETs are administered after the inhibitor is administered, therefore, it is desirable that the EETs be administered during the period in which the inhibitor will be present in amounts to be effective to delay hydrolysis of the EETs. Typically, the EET or EETs will be administered within 48 hours of administering an sEH inhibitor. Preferably, the EET or EETs are administered within 24 hours of the inhibitor, and even more preferably within 12 hours. In increasing order of desirability, the EET or EETs are administered within 10, 8, 6, 4, 2, hours, 1 hour, or one half hour after administration of the inhibitor. Most preferably, the EET or EETs are administered concurrently with the inhibitor.

In preferred embodiments, the EETs, the compound of the invention, or both, are provided in a material that permits them to be released over time to provide a longer duration of action. Slow release coatings are well known in the pharmaceutical art; the choice of the particular slow release coating is not critical to the practice of the present invention.

EETs are subject to degradation under acidic conditions. Thus, if the EETs are to be administered orally, it is desirable that they are protected from degradation in the stomach. Conveniently, EETs for oral administration may be coated to permit them to passage through the acidic environment of the stomach into the basic environment of the intestines. Such coatings are well known in the art. For example, aspirin coated with so-called “enteric coatings” is widely available commercially. Such enteric coatings may be used to protect EETs during passage through the stomach. An exemplary coating is set forth in the Examples.

While the anti-hypertensive effects of EETs have been recognized, EETs have not been administered to treat hypertension because it was thought endogenous sEH would hydrolyse the EETs too quickly for them to have any useful effect. Surprisingly, it was found during the course of the studies underlying the present invention that exogenously administered inhibitors of sEH succeeded in inhibiting sEH sufficiently that levels of EETs could be further raised by the administration of exogenous EETs. These findings underlie the co-administration of sEH inhibitors and of EETs described above with respect to inhibiting the development and progression of nephropathy. This is an important improvement in augmenting treatment. While levels of endogenous EETs are expected to rise with the inhibition of sEH activity caused by the action of the sEH inhibitor, and therefore to result in at least some improvement in symptoms or pathology, it may not be sufficient in all cases to inhibit progression of kidney damage fully or to the extent intended. This is particularly true where the diseases or other factors have reduced the endogenous concentrations of EETs below those normally present in healthy individuals. Administration of exogenous EETs in conjunction with an sEH inhibitor is therefore expected to be beneficial and to augment the effects of the sEH inhibitor in reducing the progression of diabetic nephropathy.

The present invention can be used with regard to any and all forms of diabetes to the extent that they are associated with progressive damage to the kidney or kidney function. The chronic hyperglycemia of diabetes is associated with long-term damage, dysfunction, and failure of various organs, especially the eyes, kidneys, nerves, heart, and blood vessels. The long-term complications of diabetes include retinopathy with potential loss of vision; nephropathy leading to renal failure; peripheral neuropathy with risk of foot ulcers, amputation, and Charcot joints.

In addition, persons with metabolic syndrome are at high risk of progression to type 2 diabetes, and therefore at higher risk than average for diabetic nephropathy. It is therefore desirable to monitor such individuals for microalbuminuria, and to administer an sEH inhibitor and, optionally, one or more EETs, as an intervention to reduce the development of nephropathy. The practitioner may wait until microalbuminuria is seen before beginning the intervention. Since a person can be diagnosed with metabolic syndrome without having a blood pressure of 130/85 or higher, both persons with blood pressure of 130/85 or higher and persons with blood pressure below 130/85 can benefit from the administration of sEH inhibitors and, optionally, of one or more EETs, to slow the progression of damage to their kidneys. In some preferred embodiments, the person has metabolic syndrome and blood pressure below 130/85.

Dyslipidemia or disorders of lipid metabolism is another risk factor for heart disease. Such disorders include an increased level of LDL cholesterol, a reduced level of HDL cholesterol, and an increased level of triglycerides. An increased level of serum cholesterol, and especially of LDL cholesterol, is associated with an increased risk of heart disease. The kidneys are also damaged by such high levels. It is believed that high levels of triglycerides are associated with kidney damage. In particular, levels of cholesterol over 200 mg/dL, and especially levels over 225 mg/dL, would suggest that sEH inhibitors and, optionally, EETs, should be administered. Similarly, triglyceride levels of more than 215 mg/dL, and especially of 250 mg/dL or higher, would indicate that administration of sEH inhibitors and, optionally, of EETs, would be desirable. The administration of compounds of the present invention with or without the EETs, can reduce the need to administer statin drugs (HMG-COA reductase inhibitors) to the patients, or reduce the amount of the statins needed. In some embodiments, candidates for the methods, uses, and compositions of the invention have triglyceride levels over 215 mg/dL and blood pressure below 130/85. In some embodiments, the candidates have triglyceride levels over 250 mg/dL and blood pressure below 130/85. In some embodiments, candidates for the methods, uses and compositions of the invention have cholesterol levels over 200 mg/dL and blood pressure below 130/85. In some embodiments, the candidates have cholesterol levels over 225 mg/dL and blood pressure below 130/85.

### Methods of Inhibiting the Proliferation of Vascular Smooth Muscle Cells:

In other embodiments, compounds of any one of Formula (I)-(III) and (IVa)-(IVc) or of Table 1 inhibit proliferation of vascular smooth muscle (VSM) cells without significant cell toxicity, (e.g. specific to VSM cells). Because VSM cell proliferation is an integral process in the pathophysiology of atherosclerosis, these compounds are suitable for slowing or inhibiting atherosclerosis. These compounds are useful to subjects at risk for atherosclerosis, such as individuals who have diabetes and those who have had a heart attack or a test result showing decreased blood circulation to the heart. The conditions of therapeutic administration are as described above.

The methods of the invention are particularly useful for patients who have had percutaneous intervention, such as angioplasty to reopen a narrowed artery, to reduce or to slow the narrowing of the reopened passage by restenosis. In some preferred embodiments, the artery is a coronary artery. The compounds of the invention can be placed on stents in polymeric coatings to provide a controlled localized release to reduce restenosis. Polymer compositions for implantable medical devices, such as stents, and methods for embedding agents in the polymer for controlled release, are known in the art and taught, for example, in U.S. Pat. Nos. 6,335,029; 6,322,847; 6,299,604; 6,290,722; 6,287,285; and 5,637,113. In preferred embodiments, the coating releases the inhibitor over a period of time, preferably over a period of days, weeks, or months. The particular polymer or other coating chosen is not a critical part of the present invention.

The methods of the invention are useful for slowing or inhibiting the stenosis or restenosis of natural and synthetic vascular grafts. As noted above in connection with stents, desirably, the synthetic vascular graft comprises a material which releases a compound of the invention over time to slow or inhibit VSM proliferation and the consequent stenosis of the graft. Hemodialysis grafts are a particularly preferred embodiment.

In addition to these uses, the methods of the invention can be used to slow or to inhibit stenosis or restenosis of blood vessels of persons who have had a heart attack, or whose test results indicate that they are at risk of a heart attack.

Removal of a clot such as by angioplasty or treatment with tissue plasminogen activator (tPA) can also lead to reperfusion injury, in which the resupply of blood and oxygen to hypoxic cells causes oxidative damage and triggers inflammatory events. In some embodiments, provided are methods for administering the compounds and compositions of the invention for treating reperfusion injury. In some such embodiments, the compounds and compositions are administered prior to or following angioplasty or administration of tPA.

In one group of preferred embodiments, compounds of the invention are administered to reduce proliferation of VSM cells in persons who do not have hypertension. In another group of embodiments, compounds of the invention are used to reduce proliferation of VSM cells in persons who are being treated for hypertension, but with an agent that is not an sEH inhibitor.

The compounds of the invention can be used to interfere with the proliferation of cells which exhibit inappropriate cell cycle regulation. In one important set of embodiments, the cells are cells of a cancer. The proliferation of such cells can be slowed or inhibited by contacting the cells with a compound of the invention. The determination of whether a particular compound of the invention can slow or inhibit the proliferation of cells of any particular type of cancer can be determined using assays routine in the art.

In addition to the use of the compounds of the invention, the levels of EETs can be raised by adding EETs. VSM cells contacted with both an EET and a compound of the invention exhibited slower proliferation than cells exposed to either the EET alone or to the compound of the invention alone. Accordingly, if desired, the slowing or inhibition of VSM cells of a compound of the invention can be enhanced by adding an EET along with a compound of the invention. In the case of stents or vascular grafts, for example, this can conveniently be accomplished by embedding the EET in a coating along with a compound of the invention so that both are released once the stent or graft is in position.

### Methods of Inhibiting the Progression of Obstructive Pulmonary Disease, Interstitial Lung Disease, or Asthma:

Chronic obstructive pulmonary disease, or COPD, encompasses two conditions, emphysema and chronic bronchitis, which relate to damage caused to the lung by air pollution, chronic exposure to chemicals, and tobacco smoke. Emphysema as a disease relates to damage to the alveoli of the lung, which results in loss of the separation between alveoli and a consequent reduction in the overall surface area available for gas exchange. Chronic bronchitis relates to irritation of the bronchioles, resulting in excess production of mucin, and the consequent blocking by mucin of the airways leading to the alveoli. While persons with emphysema do not necessarily have chronic bronchitis or vice versa, it is common for persons with one of the conditions to also have the other, as well as other lung disorders.

Some of the damage to the lungs due to COPD, emphysema, chronic bronchitis, and other obstructive lung disorders can be inhibited or reversed by administering inhibitors of the enzyme known as soluble epoxide hydrolase, or “sEH”. The effects of sEH inhibitors can be increased by also administering EETs. The effect is at least additive over administering the two agents separately, and may indeed be synergistic.

The studies reported herein show that EETs can be used in conjunction with sEH inhibitors to reduce damage to the lungs by tobacco smoke or, by extension, by occupational or environmental irritants. These findings indicate that the co-administration of sEH inhibitors and of EETs can be used to inhibit or slow the development or progression of COPD, emphysema, chronic bronchitis, or other chronic obstructive lung diseases which cause irritation to the lungs.

Animal models of COPD and humans with COPD have elevated levels of immunomodulatory lymphocytes and neutrophils. Neutrophils release agents that cause tissue damage and, if not regulated, will over time have a destructive effect. Without wishing to be bound by theory, it is believed that reducing levels of neutrophils reduces tissue damage contributing to obstructive lung diseases such as COPD, emphysema, and chronic bronchitis. Administration of sEH inhibitors to rats in an animal model of COPD resulted in a reduction in the number of neutrophils found in the lungs. Administration of EETs in addition to the sEH inhibitors also reduced neutrophil levels. The reduction in neutrophil levels in the presence of sEH inhibitor and EETs was greater than in the presence of the sEH inhibitor alone.

While levels of endogenous EETs are expected to rise with the inhibition of sEH activity caused by the action of the sEH inhibitor, and therefore to result in at least some improvement in symptoms or pathology, it may not be sufficient in all cases to inhibit progression of COPD or other pulmonary diseases. This is particularly true where the diseases or other factors have reduced the endogenous concentrations of EETs below those normally present in healthy individuals. Administration of exogenous EETs in conjunction with an sEH inhibitor is therefore expected to augment the effects of the sEH inhibitor in inhibiting or reducing the progression of COPD or other pulmonary diseases.

In addition to inhibiting or reducing the progression of chronic obstructive airway conditions, the invention also provides new ways of reducing the severity or progression of chronic restrictive airway diseases. While obstructive airway diseases tend to result from the destruction of the lung parenchyma, and especially of the alveoli, restrictive diseases tend to arise from the deposition of excess collagen in the parenchyma. These restrictive diseases are commonly referred to as “interstitial lung diseases”, or “ILDs”, and include conditions such as idiopathic pulmonary fibrosis. The methods, compositions, and uses of the invention are useful for reducing the severity or progression of ILDs, such as idiopathic pulmonary fibrosis. Macrophages play a significant role in stimulating interstitial cells, particularly fibroblasts, to lay down collagen. Without wishing to be bound by theory, it is believed that neutrophils are involved in activating macrophages, and that the reduction of neutrophil levels found in the studies reported herein demonstrate that the methods and uses of the invention will also be applicable to reducing the severity and progression of ILDs.

In some preferred embodiments, the ILD is idiopathic pulmonary fibrosis. In other preferred embodiments, the ILD is one associated with an occupational or environmental exposure. Exemplars of such ILDs, are asbestosis, silicosis, coal worker's pneumoconiosis, and berylliosis. Further, occupational exposure to any of a number of inorganic dusts and organic dusts is believed to be associated with mucus hypersecretion and respiratory disease, including cement dust, coke oven emissions, mica, rock dusts, cotton dust, and grain dust (for a more complete list of occupational dusts associated with these conditions, see Table 254-1 of Speizer, “Environmental Lung Diseases,” Harrison's Principles of Internal Medicine, infra, at pp. 1429-1436). In other embodiments, the ILD is sarcoidosis of the lungs. ILDs can also result from radiation in medical treatment, particularly for breast cancer, and from connective tissue or collagen diseases such as rheumatoid arthritis and systemic sclerosis. It is believed that the methods, uses and compositions of the invention can be useful in each of these interstitial lung diseases.

In another set of embodiments, the invention is used to reduce the severity or progression of asthma. Asthma typically results in mucin hypersecretion, resulting in partial airway obstruction. Additionally, irritation of the airway results in the release of mediators which result in airway obstruction. While the lymphocytes and other immunomodulatory cells recruited to the lungs in asthma may differ from those recruited as a result of COPD or an ILD, it is expected that the invention will reduce the influx of immunomodulatory cells, such as neutrophils and eosinophils, and ameliorate the extent of obstruction. Thus, it is expected that the administration of sEH inhibitors, and the administration of sEH inhibitors in combination with EETs, will be useful in reducing airway obstruction due to asthma.

In each of these diseases and conditions, it is believed that at least some of the damage to the lungs is due to agents released by neutrophils which infiltrate into the lungs. The presence of neutrophils in the airways is thus indicative of continuing damage from the disease or condition, while a reduction in the number of neutrophils is indicative of reduced damage or disease progression. Thus, a reduction in the number of neutrophils in the airways in the presence of an agent is a marker that the agent is reducing damage due to the disease or condition, and is slowing the further development of the disease or condition. The number of neutrophils present in the lungs can be determined by, for example, bronchoalveolar lavage.

### Prophylactic and Therapeutic Methods to Reduce Stroke Damage:

Inhibitors of soluble epoxide hydrolase (“sEH”) and EETs administered in conjunction with inhibitors of sEH have been shown to reduce brain damage from strokes. Based on these results, we expect that inhibitors of sEH taken prior to an ischemic stroke will reduce the area of brain damage and will likely reduce the consequent degree of impairment. The reduced area of damage should also be associated with a faster recovery from the effects of the stroke.

While the pathophysiologies of different subtypes of stroke differ, they all cause brain damage. Hemorrhagic stroke differs from ischemic stroke in that the damage is largely due to compression of tissue as blood builds up in the confined space within the skull after a blood vessel ruptures, whereas in ischemic stroke, the damage is largely due to loss of oxygen supply to tissues downstream of the blockage of a blood vessel by a clot. Ischemic strokes are divided into thrombotic strokes, in which a clot blocks a blood vessel in the brain, and embolic strokes, in which a clot formed elsewhere in the body is carried through the blood stream and blocks a vessel there. In both hemorrhagic stroke and ischemic stroke, the damage is due to the death of brain cells. Based on the results observed in our studies, we would expect at least some reduction in brain damage in all types of stroke and in all subtypes.

A number of factors are associated with an increased risk of stroke. Given the results of the studies underlying the present invention, sEH inhibitors administered to persons with any one or more of the following conditions or risk factors: high blood pressure, tobacco use, diabetes, carotid artery disease, peripheral artery disease, atrial fibrillation, transient ischemic attacks (TIAs), blood disorders such as high red blood cell counts and sickle cell disease, high blood cholesterol, obesity, alcohol use of more than one drink a day for women or two drinks a day for men, use of cocaine, a family history of stroke, a previous stroke or heart attack, or being elderly, will reduce the area of brain damaged by a stroke. With respect to being elderly, the risk of stroke increases for every 10 years. Thus, as an individual reaches 60, 70, or 80, administration of sEH inhibitors has an increasingly larger potential benefit. As noted in the next section, the administration of EETs in combination with one or more sEH inhibitors can be beneficial in further reducing the brain damage.

In some preferred uses and methods, the sEH inhibitors and, optionally, EETs, are administered to persons who use tobacco, have carotid artery disease, have peripheral artery disease, have atrial fibrillation, have had one or more transient ischemic attacks (TIAs), have a blood disorder such as a high red blood cell count or sickle cell disease, have high blood cholesterol, are obese, use alcohol in excess of one drink a day if a woman or two drinks a day if a man, use cocaine, have a family history of stroke, have had a previous stroke or heart attack and do not have high blood pressure or diabetes, or are 60, 70, or 80 years of age or more and do not have hypertension or diabetes.

Clot dissolving agents, such as tissue plasminogen activator (tPA), have been shown to reduce the extent of damage from ischemic strokes if administered in the hours shortly after a stroke. For example, tPA is approved by the FDA for use in the first three hours after a stroke. Thus, at least some of the brain damage from a stoke is not instantaneous, but rather occurs over a period of time or after a period of time has elapsed after the stroke. It is contemplated that administration of sEH inhibitors, optionally with EETs, can also reduce brain damage if administered within 6 hours after a stroke has occurred, more preferably within 5, 4, 3, or 2 hours after a stroke has occurred, with each successive shorter interval being more preferable. Even more preferably, the inhibitor or inhibitors are administered 2 hours or less or even 1 hour or less after the stroke, to maximize the reduction in brain damage. Persons of skill are well aware of how to make a diagnosis of whether or not a patient has had a stroke. Such determinations are typically made in hospital emergency rooms, following standard differential diagnosis protocols and imaging procedures.

In some preferred uses and methods, the sEH inhibitors and, optionally, EETs, are administered to persons who have had a stroke within the last 6 hours who: use tobacco, have carotid artery disease, have peripheral artery disease, have atrial fibrillation, have had one or more transient ischemic attacks (TIAs), have a blood disorder such as a high red blood cell count or sickle cell disease, have high blood cholesterol, are obese, use alcohol in excess of one drink a day if a woman or two drinks a day if a man, use cocaine, have a family history of stroke, have had a previous stroke or heart attack and do not have high blood pressure or diabetes, or are 60, 70, or 80 years of age or more and do not have hypertension or diabetes.

### Combination Therapy

As noted above, the compounds of the present invention will, in some instances, be used in combination with other therapeutic agents to bring about a desired effect. Selection of additional agents will, in large part, depend on the desired target therapy (see, e.g., Turner, N. et al. Prog. Drug Res. (1998) 51: 33-94; Haffner, S. Diabetes Care (1998) 21: 160-178; and DeFronzo, R. et al. (eds), Diabetes Reviews (1997) Vol. 5 No. 4). A number of studies have investigated the benefits of combination therapies with oral agents (see, e.g., Mahler, R., J. Clin. Endocrinol. Metab. (1999) 84: 1165-71; United Kingdom Prospective Diabetes Study Group: UKPDS 28, Diabetes Care (1998) 21: 87-92; Bardin, C. W., (ed), Current Therapy In Endocrinology And Metabolism, 6th Edition (Mosby-Year Book, Inc., St. Louis, Mo. 1997); Chiasson, J. et al., Ann. Intern. Med. (1994) 121: 928-935; Coniff, R. et al., Clin. Ther. (1997) 19: 16-26; Coniff, R. et al., Am. J. Med. (1995) 98: 443-451; and Iwamoto, Y. et al., Diabet. Med. (1996) 13 365-370; Kwiterovich, P. Am. J. Cardiol (1998) 82(12A): 3U-17U). Combination therapy includes administration of a single pharmaceutical dosage formulation which contains a compound of any one of Formula (I)-(III) and (IVa)-(IVc) or of Table 1 and one or more additional active agents, as well as administration of the compound and each active agent in its own separate pharmaceutical dosage formulation. For example, a compound of any one of Formula (I)-(III) and (IVa)-(IVc) or of Table 1 and one or more angiotensin receptor blockers, angiotensin converting enzyme inhibitors, calcium channel blockers, diuretics, alpha blockers, beta blockers, centrally acting agents, vasopeptidase inhibitors, renin inhibitors, endothelin receptor agonists, AGE (advanced glycation end-products) crosslink breakers, sodium/potassium ATPase inhibitors, endothelin receptor agonists, endothelin receptor antagonists, angiotensin vaccine, and the like; can be administered to the human subject together in a single oral dosage composition, such as a tablet or capsule, or each agent can be administered in separate oral dosage formulations. Where separate dosage formulations are used, the compound of any one of Formula (I)-(III) and (IVa)-(IVc) or of Table 1 and one or more additional active agents can be administered at essentially the same time (i.e., concurrently), or at separately staggered times (i.e., sequentially). Combination therapy is understood to include all these regimens.

### Administration and Pharmaceutical Composition

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors. The drug can be administered more than once a day, preferably once or twice a day. All of these factors are within the skill of the attending clinician.

Therapeutically effective amounts of the compounds may range from approximately 0.05 to 50 mg per kilogram body weight of the recipient per day; preferably about 0.1-25 mg/kg/day, more preferably from about 0.5 to 10 mg/kg/day. Thus, for administration to a 70 kg person, the dosage range would most preferably be about 35-70 mg per day.

In general, compounds of this invention will be administered as pharmaceutical compositions by any one of the following routes: oral, systemic (e.g., transdermal, intranasal or by suppository), parenteral (e.g., intramuscular, intravenous or subcutaneous), or intrathecal administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of this invention is inhalation. This is an effective method for delivering a therapeutic agent directly to the respiratory tract (see U.S. Pat. No. 5,607,915).

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical formulations have been developed especially for drugs that show poor bioavailability based upon the principle that bioavailability can be increased by increasing the surface area, i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of the invention in combination with at least one pharmaceutically acceptable excipient. Acceptable excipients are non-toxic, aid administration, and do not adversely affect the therapeutic benefit of the compound. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of the compound of based on the total formulation, with the balance being one or more suitable pharmaceutical excipients. Preferably, the compound is present at a level of about 1-80 wt %. Representative pharmaceutical formulations containing a compound of any one of Formula (I)-(III) and (IVa)-(IVc) or of Table 1 are described below.

### General Synthetic Methods

The compounds of this invention can be prepared from readily available starting materials using the following general methods and procedures. It will be appreciated that where typical or preferred process conditions (i.e., reaction temperatures, times, mole ratios of reactants, solvents, pressures, etc) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the compounds of this invention may contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4^{th} Edition), and Larock's Comprehensive Organic Transformations (VCH Publishers Inc., 1989).

The various starting materials, intermediates, and compounds of the invention may be isolated and purified where appropriate using conventional techniques such as precipitation, filtration, crystallization, evaporation, distillation, and chromatography. Characterization of these compounds may be performed using conventional methods such as by melting point, mass spectrum, nuclear magnetic resonance, and various other spectroscopic analyses.

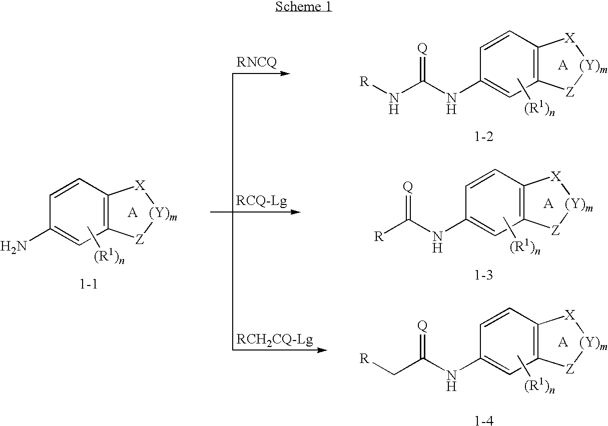

A synthesis of the compounds of the invention is shown in Scheme 1, where A, Q, R, X, Y, Z, R¹, m, and n are previously defined, and where Lg is a leaving group such as halogen. Compound 1-1 can be used as a starting material to form a variety of compounds having a urea, thiourea, amide or thioamide linkage.

Reaction of amine 1-1 with isocyanate or isothiocyanate RNCQ gives the corresponding urea or thiourea 1-2. Typically, the preparation of the urea is conducted using a polar solvent such as DMF (dimethylformamide) at 60 to 85° C.

Reactions of 1-3 with RCQ-Lg or RCH₂CQ-Lg where Lg is OH or a leaving group such as halogen under amide forming conditions can form the respective amides/thioamides 1-3 and 1-4.

When Lg is OH, a variety of amide coupling reagents may be used to from the amide bond, including the use of carbodiimides such as N—N′-dicyclohexylcarbodiimide (DCC), N—N′-diisopropylcarbodiimide (DIPCDI), and 1-ethyl-3-(3′-dimethylaminopropyl)carbodiimide (EDCI). The carbodiimides may be used in conjunction with additives such as dimethylaminopyridine (DMAP) or benzotriazoles such as 7-aza-1-hydroxybenzotriazole (HOAt), 1-hydroxybenzotriazole (HOBt), and 6-chloro-1-hydroxybenzotriazole (Cl—HOBt).

Amide coupling reagents also include amininum and phosphonium based reagents. Aminium salts include N-[(dimethylamino)-1H-1,2,3-triazolo[4,5-b]pyridine-1-ylmethylene]-N-methylmethanaminium hexafluorophosphate N-oxide (HATU), N-[(1H-benzotriazol-1-yl)(dimethylamino)methylene]-N-methylmethanaminium hexafluorophosphate N-oxide (HBTU), N-[(1H-6-chlorobenzotriazol-1-yl)(dimethylamino)methylene]-N-methylmethanaminium hexafluorophosphate N-oxide (HCTU), N-[(1H-benzotriazol-1-yl)(dimethylamino)methylene]-N-methylmethanaminium tetrafluoroborate N-oxide (TBTU), and N-[(1H-6-chlorobenzotriazol-1-yl)(dimethylamino)methylene]-N-methylmethanaminium tetrafluoroborate N-oxide (TCTU). Phosphonium salts include 7-azabenzotriazol-1-yl-N-oxy-tris(pyrrolidino)phosphonium hexafluorophosphate (PyAOP) and benzotriazol-1-yl-N-oxy-tris(pyrrolidino)phosphonium hexafluorophosphate (PyBOP). Amide formation step may be conducted in a polar solvent such as dimethylformamide (DMF) and may also include an organic base such as diisopropylethylamine (DIEA) or dimethylaminopyridine (DMAP).

Generally, amine 1-1 may be readily available from commercial sources or prepared by conventional methods and procedures known to a person of skill in the art. For example, amine 1-1 may be prepared from the corresponding nitro compound 2-1 by reduction by a reducing agent, as shown in Scheme 2. Suitable reducing agents to effect this transformation include hydrogenation in the presence of a catalyst such as Ni or Pd or treatment of 1-1 with iron and an acid such as ammonium formate.

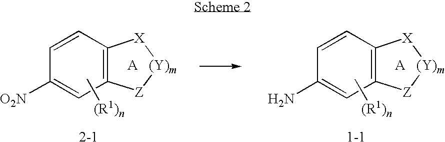

The following examples are provided to illustrate certain aspects of the present invention and to aid those of skill in the art in practicing the invention. These examples are in no way to be considered to limit the scope of the invention.

### EXAMPLES

The examples below as well as throughout the application, the following abbreviations have the following meanings. If not defined, the terms have their generally accepted meanings.
aq.=aqueous
brs=broad singlet
d=doublet
DCM=dichloromethane
DMAP=dimethylaminopyridine
DMF=dimethylformamide
DMSO=dimethylsulfoxide
equiv.=equivalent
EtOAc=ethyl acetate
g=gram
h=hour
LCMS=liquid chromatography mass spectroscopy
m=multiplet
MHz=megahertz
mL=milliliter
m.p.=melting point
N=normal
s=singlet
t=triplet
TLC=thin layer chromatography

### Example 1

### 1-(2,2-difluorobenzo[1,3]dioxol-5-yl)-3-(4-(trifluoromethyl)phenyl)urea (3)

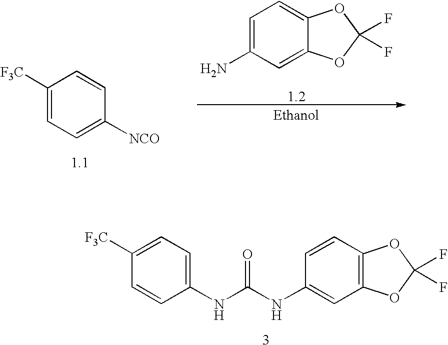

Isocyanate 1.1 (1 equiv.) was added to a solution of amine 1.2 (1 equiv.) in ethanol, and the reaction mixture was warmed to 70° C. and stirred at this temperature overnight. The solvent was then removed in vacuo, and the crude product was triturated with diethyl ether to give compound 3 as a pale pink powder. M.P.: 195-200° C.; Mass 361 [M+1]; ¹H NMR (400 MHz; DMSO-d₆) δ: 7.0-7.2 (d, 1H, ArCH); 7.3-7.4 (d, 1H, ArCH); 7.6-7.8 (m, 5H, ArCH); 8.9-9.2 (s, 2H, NH); LCMS purity: 98.7%; Yield: 80.5%.

### Example 2

### 1-(1-oxo-1,3-dihydroisobenzofuran-5-yl)-3-(4-(trifluoromethyl)phenyl)urea (18)

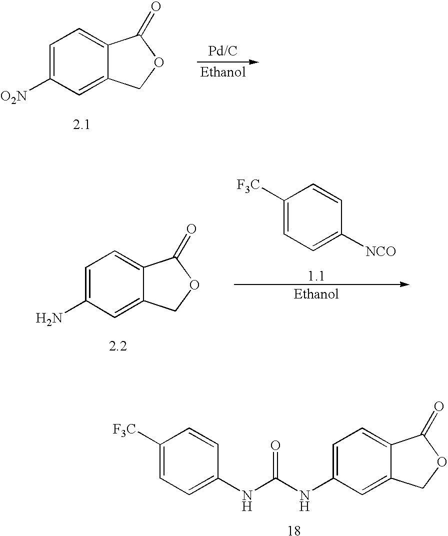

Compound 2.1 (1 equiv.) was dissolved in absolute ethanol and hydrogenated over 10% palladium on carbon at atmospheric pressure at room temperature overnight. The mixture was then filtered, concentrated in vacuo to give the crude product which was triturated with diethyl ether to give compound 2.2 as a solid.

Isocyanate 1.1 (1 equiv.) was added to a solution of amine 2.2 (1 equiv.) in ethanol, and the reaction mass was warmed to 70° C. and stirred at this temperature overnight. The solvent was removed in vacuo and the crude product was recrystallised in diethyl ether to give the compound as a light brown powder. M.P.: 297-307° C.; Mass 335 [M+1]; ¹H NMR (400 MHz; DMSO-d₆) δ: 5.2-5.4 (s, 2H, CH2); 7.4-7.6 (d, 1H, ArCH); 7.6-7.7 (t, 4H, ArCH); 7.7-7.8 (d, 1H, ArCH); 7.8-7.9 (s, 1H, ArCH); 9.2-9.4 (s, 1H, NH); 9.4-9.6 (s, 1H, NH)); LCMS purity: 97.8%; Yield: 55.5%.

### Example 3

### 1-(1-oxo-1,3-dihydroisobenzofuran-5-yl)-3-(3-(trifluoroethyl)phenyl)urea (17)

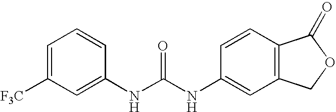

The title compound was prepared using a procedure similar to that described in Example 2 as a light brown powder, using the amine 2.2 and 3-trifluoromethyl phenyl isocyanate. M.P.: 279-282° C.; Mass 335 [M+1]; ¹H NMR (400 MHz; DMSO-d₆) δ: 5.2-5.4 (s, 2H, CH2); 7.2-7.4 (d, 1H, ArCH); 7.5-7.6 (t, 2H, ArCH); 7.6-7.7 (d, 1H, ArCH); 7.7-7.8 (d, 1H, ArCH); 7.9-8.0 (s, 1H, ArCH); 8.0-8.1 (s, 1H, ArCH); 9.0-9.2 (s, 1H, NH); 9.2-9.4 (s, 1H, NH); LCMS purity: 94.6%; Yield: 55.5%.

### Example 4

### 1-(1-methyl-1H-indazol-5-yl)-3-(4-(trifluoromethyl)phenyl)urea (8)

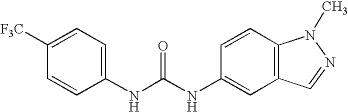

### Synthesis of 1-methyl-5-nitro-1H-indazole (3.2)

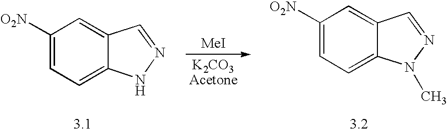

To a solution of compound 3.1 (1 equiv) in acetone was added potassium carbonate (3 equiv.) and iodomethane (1.5 equiv.) with stirring at 0° C., and the resulting mixture was warmed to reflux and maintained at this temperature overnight. The reaction mass concentrated in vacuo, and the residue treated with water and extracted with ethyl acetate. The organic layer was separated, washed with water and brine, dried over sodium sulfate, and concentrated to give the crude product which was then purified by silica gel column chromatography to obtain compound 3.2 as a solid.

### Synthesis of 1-(1-methyl-1H-indazol-5-yl)-3-(4-(trifluoromethyl)phenyl)urea (8)

Using a procedure similar to that described in Example 2, compound 3.2 was converted to the corresponding aniline and then coupled with isocyanate 1.1 to give Compound 8 as a greenish blue powder. M.P.: 252-254° C.; Mass 335 [M+1]; ¹H NMR (400 MHz; DMSO-d₆) δ: 4.0-4.1 (s, 3H, CH3); 7.3-7.4 (d, 1H, ArCH); 7.5-7.6 (d, 1H, ArCH); 7.6-7.7 (m, 4H, ArCH); 7.8-8.0 (s, 2H, ArCH); 8.7-8.8 (s, 1H, NH); 9.0-9.1 (s, 1H, NH); LCMS purity: 98.1%; Yield: 53%.

### Example 5

### 1-adamantan-1-yl-3-(2,3-dihydrobenzo[b][1,4]dioxin-6-yl)urea (2)

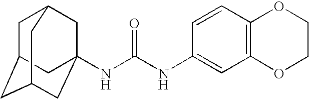

The title compound was prepared as a light brown powder using a procedure similar to that described in Example 1. M.P.: 230-236° C., Mass 329 [M+1]; ¹H NMR (300 MHz; DMSO-d₆) δ: 1.6-1.7 (6H, m, CH); 1.9-2.1 (m, 9H, CH); 4.2-4.4 (s, OCH2); 4.5 (s, 1H, NH); 6.1 (s, 1H, NH); 6.8 (dd, 1H, ArCH); 6.9-7.0 (d, 2H, ArCH); LCMS purity: 97.3%; Yield: 70%.

### Example 6

### 1-adamantan-1-yl-3-(2,2-difluorobenzo[d][1,3]dioxol-5-yl)urea (10)

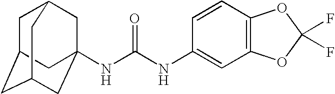

The title compound was prepared as a off-white powder using a procedure similar to that described in Example 1. M.P.: 213-215° C.; Mass 351 [M+1]; ¹H NMR (400 MHz; DMSO-d₆) δ: 1.6-1.7 (m , 6H, CH); 1.9-2.1 (m, 9H, CH); 5.8-6.0 (s, 1H, NH); 6.8-7.0 (d, 1H, Ar—CH); 7.0-7.2 (d, 1H, ArCH); 7.5-7.6 (dd, 1H, ArCH); 8.4-8.5 (s, 1H, NH); LCMS purity: 99.6%; Yield: 67%.

### Example 7

### 1-adamantan-1-yl-3-(2-methyl-1H-indol-5-yl)urea (9)

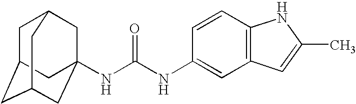

The title compound was prepared as an off-white solid using a procedure similar to that described in Example 2. M.P.: 146-152° C.; Mass 324 [M+1]; ¹H NMR (400 MHz; DMSO-d₆) δ: 1.6-1.7 (m, 6H, CH, CH2); 1.9-2.1 (m, 9H, CH, CH2); 2.3-2.4 (s, 3H, CH3); 5.7-5.8 (s, 1H, NH); 6.0-6.1 (s, 1H, NH); 6.8-6.9 (dd, 1H, ArCH); 7.0-7.1 (dd, 1H, ArCH); 7.4-7.5 (s, 1H, ArCH); 7.8-8.0 (s, 1H, ArCH); 10.6-10.7 (s, 1H, NH); LCMS purity: 97.6%; Yield: 60%.

### Example 8

### 1-adamantan-1-yl-3-(2-(trifluoromethyl)-1H-benzo[d]imidazol-5-yl)urea (15)

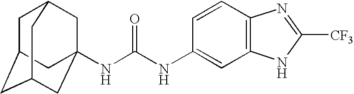

The title compound was prepared as a light brown powder using a procedure similar to that described in Example 2. M.P.: 189-192° C.; Mass 379 [M+1]; ¹H NMR (400 MHz; DMSO-d₆+CDCl₃) δ: 1.6-1.8 (m, 7H, CH, CH2); 2.0-2.2 (m, 10H, CH, CH2); 7.0-7.2 (d, 1H, ArCH); 7.4-7.6 (d, 1H, ArCH); 8.0-8.05 (s, 1H, ArCH); 8.2-8.4 (s, 1H, NH); LCMS purity: 99.4%; Yield: 47%.

### Example 9

### 1-adamantan-1-yl-3-(2-methylbenzo[d]thiazol-5-yl)urea (13)

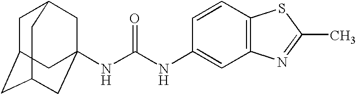

The title compound was prepared as a light brown powder using a procedure similar to that described in Example 1. M.P.: 209-214° C.; Mass 342 [M+1]; ¹H NMR (400 MHz; DMSO-d₆) δ: 1.62-1.7 (m, 6H, CH, CH2); 1.92-2.1 (m, 9H, CH, CH2); 2.7-2.8 (s, 1H, CH3); 5.9-6.0 (s, 1NH); 7.2-7.3 (dd, 1H, ArCH); 7.8-7.9 (dd, 1H, ArCH); 8.0-8.1 (s, H, ArCH); 8.4-8.5 (s, 1H, NH); LCMS purity: 96.0%; Yield: 30%.

### Example 10

### 1-(quinolin-6-yl)-3-(4-(trifluoromethyl)phenyl)urea (1)

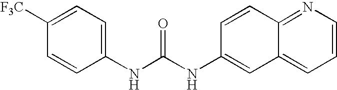

The title compound was prepared as a pale yellow powder using a procedure similar to that described in Example 1. M.P.: 259-263° C.; Mass 332 [M+1]; ¹H NMR (300 MHz; DMSO-d₆): δ: 7.6-7.8 (m, 4H, Ar—CH); 8.0-8.1 (m, 2H, ArCH); 8.3-8.4 (d, 1H, ArCH.); 8.5-8.6 (s, 1H, ArCH); 9.0-9.2 (m, 2H, Ar—CH); 10.0-10.2 (s, 1H, NH); 10.3-10.4 (s, 1H, NH); LCMS purity: 99.7%; Yield: 85.5%.

### Example 11

### 1-(2-(trifluoromethyl)-1H-benzo[d]imidazol-5-yl)-3-(4-(trifluoromethyl)phenyl)urea (14)

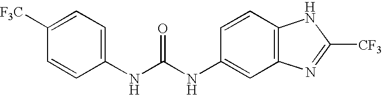

The title compound was prepared as a light brown powder using a procedure similar to that described in Example 2. M.P.: 223-228° C.; Mass 389 [M+1]; ¹H NMR (400 MHz; DMSO-d₆) δ: 7.2-7.4 (d, 1H, ArCH); 7.6-7.8 (q, 5H, ArCH); 8.0-8.2 (s, 1H, ArCH); 9.0-9.2 (s, 1H, NH); 9.2-9.4 (s, 1H, NH); LCMS purity: 99.4%; Yield: 70%.

### Example 12

### 1-(2-methylbenzo[d]oxazol-5-yl)-3-(4-(trifluoromethyl)phenyl)urea (11)

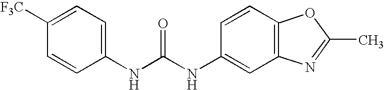

The title compound was prepared as a light brown powder using a procedure similar to that described in Example 1. M.P.: 240-243° C.; Mass 336 [M+1]; ¹H NMR (400 MHz; DMSO-d₆) δ: 2.5-2.7 (s, 3H, CH3); 7.3-7.4 (d, 1H, ArCH); 7.5-7.6 (d, 1H, ArCH); 7.6-7.7 (m, 4H, ArCH); 7.8-7.9 (s, 1H, ArCH); 8.9-9.0 (s, 1H, NH); 9.1-9.2 (s, 1H, NH); LCMS purity: 99.7%; Yield: 80%.

### Example 13

### 1-(4-chlorophenyl)-3-(2,2-difluorobenzo[d][1,3]dioxol-5-yl)urea (7)

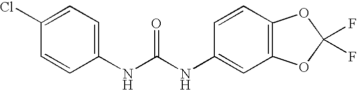

The title compound was prepared as an off-white powder using a procedure similar to that described in Example 1. M.P.: 231-233° C.; Mass 327 [M+1]; ¹H NMR (400 MHz; DMSO-d₆) δ: 7.0-7.1 (dd, 1H, ArCH); 7.3-7.4 (m, 3H, ArCH); 7.45-7.5 (d, 2H, ArCH); 7.6-7.65 (d, 1H, Ar—CH); 8.8-9.0 (s, 2H, NH); LCMS purity: 99.2%; Yield: 92%.

### Example 14

### 1-(4-chlorophenyl)-3-(2-methyl-1H-indol-5-yl)urea (6)

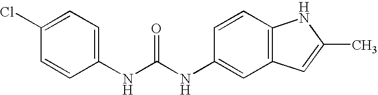

The title compound was prepared as a light brown powder using a procedure similar to that described in Example 2. M.P.: 257-260° C.; Mass 300 [M+1]; ¹H NMR (400 MHz; DMSO-d₆) δ: 2.3-2.4 (s, 3H, CH3); 6.0-6.1 (s, 1H, CH); 6.8-7.0 (dd, 1H, ArCH); 7.1-7.2 (d, 1H, ArCH); 7.3-7.32 (d, 2H, ArCH); 7.45-7.5 (t, 3H, ArCH); 8.3-8.4 (s, 1H, NH); 8.6-8.7 (s, 1H, NH); 10.8-10.9 (s, 1H, NH); LCMS purity: 99.6%; Yield: 72%.

### Example 15

### 1-(4-chlorophenyl)-3-(2-(trifluoromethyl)-1H-benzo[d]imidazol-5-yl)urea (4)

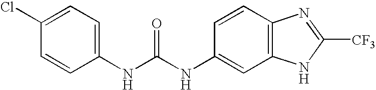

The title compound was prepared as an off-white powder using a procedure similar to that described in Example 2. M.P.: 264-268° C.; Mass 355 [M+1]; ¹H NMR (400 MHz; DMSO-d₆) δ: 7.2-7.25 (m, 1H, ArCH); 7.3-7.35 (d, 2H, ArCH); 7.5-7.55 (d, 2H, ArCH); 7.6-7.65 (d, 1H, ArCH); 8.0-8.05 (d, 1H, ArCH); 8.8-9.0 (d, 2H, NH); LCMS purity: 98.2%; Yield: 54%.

### Example 16

### 1-(2,2-difluorobenzo[d][1,3]dioxol-5-yl)-3-(3-(trifluoromethyl)phenyl)urea (5)

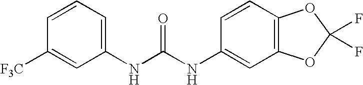

The title compound was prepared as an off-white powder using a procedure similar to that described in Example 1. M.P.: 187-191° C.; Mass 361 [M+1]; ¹H NMR (400 MHz; DMSO-d₆) δ: 7.1-7.2 (dd, 1H, ArCH); 7.3-7.4 (d, 2H, ArCH); 7.5-7.6 (m, 2H, ArCH); 7.65-7.7 (s, 1H, ArCH); 8.0-8.05 (s, 1H, ArCH); 8.9-9.1 (s, 2H, NH); LCMS purity: 98%; Yield: 89%.

### Example 17

### 1-(2-(trifluoromethyl)-1H-benzo[d]imidazol-5-yl)-3-(3-(trifluoromethyl)phenyl)urea (16)

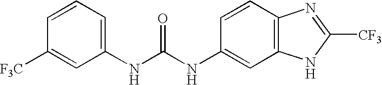

The title compound was prepared as a brown powder using a procedure similar to that described in Example 2. M.P.: 213-215° C.; Mass 389 [M+1]; ¹H NMR (400 MHz; DMSO-d₆) δ: 7.2-7.4 (d, 2H, ArCH); 7.4-7.8 (m, 3H, ArCH); 8.0-8.2 (s, 2H, ArCH); 9.0-9.4 (2s, 2H, NH); LCMS purity: 97.3%; Yield: 83%.

### Example 18

### 1-(2-methylbenzo[d]oxazol-5-yl)-3-(3-(trifluoromethyl)phenyl)urea (12)

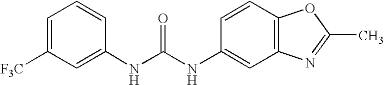

The title compound was prepared as a pale pink powder using a procedure similar to that described in Example 1. M.P.: 184-186° C.; Mass 336 [M+1]; ¹H NMR (400 MHz; DMSO-d₆) δ: 2.5-2.6 (s, 3H, CH3); 7.3-7.35 (m, 2H, ArH); 7.5-7.6 (m, 3H, ArCH); 7.8-7.9 (s, 1H, ArCH); 8.0-8.05 (s, 1H, ArCH); 8.8-9.0 (s, 2H, NH); LCMS purity: 99.6%; Yield: 90%.

### Biological Examples

### Example 1

### Fluorescent Assay for Mouse and Human Soluble Epoxide Hydrolase

Recombinant mouse sEH (MsEH) and human sEH (HsEH) were produced in a baculovirus expression system as previously reported. Grant et al., J. Biol. Chem., 268:17628-17633 (1993); Beetham et al., Arch. Biochem. Biophys., 305:197-201 (1993). The expressed proteins were purified from cell lysate by affinity chromatography. Wixtrom et al., Anal. Biochem., 169:71-80 (1988). Protein concentration was quantified using the Pierce BCA assay using bovine serum albumin as the calibrating standard. The preparations were at least 97% pure as judged by SDS-PAGE and scanning densitometry. They contained no detectable esterase or glutathione transferase activity which can interfere with the assay. The assay was also evaluated with similar results in crude cell lysates or homogenate of tissues.

The IC₅₀s for each inhibitor were according to the following procedure:

### Substrate:

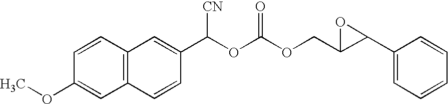

Cyano(2-methoxynaphthalen-6-yl)methyl (3-phenyloxiran-2-yl)methyl carbonate (CMNPC; Jones P. D. et. al.; Analytical Biochemistry 2005; 343: pp. 66-75)

### Solutions:

Bis/Tris HCl 25 mM pH 7.0 containing 0.1 mg/mL of BSA (buffer A)

### CMNPC at 0.25 mM in DMSO.

Mother solution of enzyme in buffer A (Mouse sEH at 6 μg/mL and Human sEH at 5 μg/mL).
Inhibitor dissolved in DMSO at the appropriate concentration.

### Protocol:

In a black 96 well plate, fill all the wells with 150 μL of buffer A.
Add 2 μL of DMSO in well A2 and A3, and then add 2 μL of inhibitor solution in A1 and A4 through A12.
Add 150 μL of buffer A in row A, then mix several time and transfer 150 μL to row B. Repeat this operation up to row H. The 150 μL removed from row H go to the trash.
Add 20 μL of buffer A in column 1 and 2, then add 20 μL of enzyme solution to column 3 to 12.
Incubate the plate for 5 minutes in the plate reader at 30° C.
During incubation prepare the working solution of substrate by mixing 3.68 mL of buffer A (4×0.920 mL) with 266 μL (2×133 μL) of substrate solution).
At t=0, add 30 μL of working substrate solution with multi-channel pipette labeled “Briggs 303” and start the reading ([S]_{final}: 5 μM).
Read with ex: 330 nm (20 nm) and em: 465 nm (20 nm) every 30 second for 10 minutes. The velocities are used to analyze and calculate the IC₅₀s.

Table 3 shows the percent inhibition (% Inh) of Compounds 1-18 when tested with the assay at 50, 500, 5000 nM.

TABLE 3

| | Conc | |
|---|---|---|
| Cmpd | (nM) | % Inh |
| 1 | 2000 | 98 |
| 2 | 2000 | 98 |
| 3 | 50 | 91 |
| 4 | 500 | 91 |
| 5 | 500 | 89 |
| 6 | 50 | 89 |
| 7 | 50 | 95 |
| 8 | 50 | 92 |
| 9 | 50 | 85 |
| 10 | 50 | 99 |
| 11 | 50 | 98 |
| 12 | 500 | 93 |
| 13 | 50 | 92 |
| 14 | 50 | 80 |
| 15 | 500 | 93 |
| 16 | 500 | 85 |
| 17 | 50 | 86 |
| 18 | 50 | 87 |

### Formulation Examples

The following are representative pharmaceutical formulations containing a compound of the present invention.

### Example 1

### Tablet Formulation

The following ingredients are mixed intimately and pressed into single scored tablets.

| Ingredient | Quantity per tablet, mg |
|---|---|
| Compound of the invention | 400 |
| Cornstarch | 50 |
| Croscarmellose sodium | 25 |
| Lactose | 120 |
| Magnesium stearate | 5 |

### Example 2

### Capsule Formulation

The following ingredients are mixed intimately and loaded into a hard-shell gelatin capsule.

| Ingredient | Quantity per tablet, mg |
|---|---|
| Compound of the invention | 200 |
| Lactose, spray-dried | 148 |
| Magnesium stearate | 2 |

### Example 3

### Suspension Formulation

The following ingredients are mixed to form a suspension for oral administration (q.s.=sufficient amount).

| Ingredient | Amount | |
|---|---|---|
| Compound of the invention | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.0 | g |
| Sorbitol (70% solution) | 13.0 | g |
| Veegum K (Vanderbilt Co) | 1.0 | g |
| flavoring | 0.035 | mL |
| colorings | 0.5 | mg |
| distilled water | q.s. to 100 | mL |

### Example 4

### Injectable Formulation

The following ingredients are mixed to form an injectable formulation.

| Ingredient | Quantity per tablet, mg |
|---|---|
| Compound of the invention | 0.2 mg-20 mg |
| sodium acetate buffer solution, 0.4 M | 2.0 mL |
| HCl (1N) or NaOH (1N) | q.s. to suitable pH |
| water (distilled, sterile) | q.s. to 20 mL |

### Example 5

### Suppository Formulation

A suppository of total weight 2.5 g is prepared by mixing the compound of the invention with Witepsol® H-15 (triglycerides of saturated vegetable fatty acid; Riches-Nelson, Inc., New York), and has the following composition:

| Ingredient | Quantity per tablet, mg |
|---|---|
| Compound of the invention | 500 mg |
| Witepsol ® H-15 | balance |

1. A compound of Formula (I) or a pharmaceutically acceptable salt thereof: 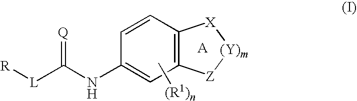
wherein:
A is an optionally substituted cycloalkyl, optionally substituted heterocyclic or optionally substituted heteroaryl ring fused with the benzene ring;
each of X, Y and Z are independently selected from the group consisting of —CR═, —C(R⁴)(R⁵)—, —C(═O)—, —C(═S)—, —N═, —NR⁶—, —O—, —S—, —SO—, and —SO₂—, provided that at least one of X, Y and Z is selected from the group consisting of —C(═O)—, —C(═S)—, —N═, —NR⁶—, —O—, —S—, —SO—, and —SO₂—; and provided that X and Z are not both C(═O) and that A is not substituted with four fluoro;
wherein
each R³, R⁴ and R⁵ is independently selected from the group consisting of R⁶, halo, alkoxy, substituted alkoxy, acyloxy, carboxyl ester, acylamino, alkylamino, aminocarbonylamino, aminocarbonyloxy, aminosulfonylamino, (substituted sulfonyl)amino, (substituted sulfonyl)aminocarbonyl, (carboxyl ester)amino;
R⁶ is independently selected from the group consisting of hydrogen, alkyl, substituted alkyl, alkenyl, substituted alkenyl, alkynyl, substituted alkynyl, heteroaryl, heterocyclyl, haloalkyl, acyl, aminosulfonyl, and aminocarbonyl;
m is 1, 2 or 3;
R is selected from the group consisting of C₆₋₁₀ cycloalkyl, substituted C₆₋₁₀ cycloalkyl and 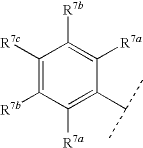
wherein each R^{7a} is independently hydrogen or halo;
each of R^{7b} and R^{7c} is independently selected from the group consisting of hydrogen, halo, alkyl, acyl, acyloxy, carboxyl ester, acylamino, alkylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, alkylsulfonyl and haloalkylsulfonyl; or R^{7c} and one of R^{7b} together form a heterocycloalkyl ring fused with the phenyl; provided that if one of R^{7b} is hydrogen and the other R^{7b} is halo, then R^{7c} and is not the same halo;
each R¹ is independently selected from the group consisting of alkyl, cyano, halo, and haloalkyl;
n is 0, 1, 2, or 3;
Q is O or S;
L is a covalent bond, —NH—, or —CR′R″— where R′ and R″ are independently H or alkyl or R′ and R″ together form a C₃-C₆ cycloalkyl ring;
with the proviso that when L is —NH—, m is 1, and X, Y and Z with the phenyl ring define an indole or benzimidazole, then at least one of X, Y, or Z is selected from —NR⁶— and —CR³═ where R⁶ or R³ is not hydrogen; and
with the proviso that Formula (I) is not:

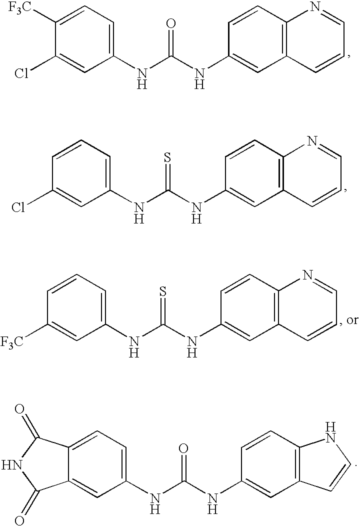

2-3. (canceled)

4. A compound of claim 1 of Formula (II) or a pharmaceutically acceptable salt thereof: 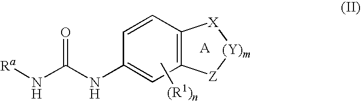
wherein:
A is an optionally substituted cycloalkyl, optionally substituted heterocyclic or optionally substituted heteroaryl ring fused with the benzene ring;
each of X, Y and Z are independently selected from the group consisting of —CR³═, —C(R⁴)(R⁵)—, —C(═O)—, —C(═S)—, ═N—, —NR⁶—, —O—, —S—, —SO—, and —SO₂—, provided that at least one of X, Y and Z is selected from the group consisting of —C(═O)—, —C(═S)—, —N═, —NR⁶—, —O—, —S—, —SO—, and —SO₂—; and provided that X and Z are not both —C(═O)— and that A is not substituted with four fluoro;
wherein
each R³, R⁴ and R⁵ is independently selected from the group consisting of R⁶, halo, alkoxy, substituted alkoxy, acyloxy, carboxyl ester, acylamino, alkylamino, aminocarbonylamino, aminocarbonyloxy, aminosulfonylamino, (substituted sulfonyl)amino, (substituted sulfonyl)aminocarbonyl, (carboxyl ester)amino;
R⁶ is independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclyl, substituted heterocyclyl, haloalkyl, acyl, aminosulfonyl, and aminocarbonyl;
m is 1, 2 or 3;
each R¹ is independently selected from the group consisting of alkyl, cyano, halo, and haloalkyl;
n is 0, 1, 2, or 3;
R^{a} is selected from the group consisting of C₆₋₁₀ cycloalkyl, substituted C₆₋₁₀ cycloalkyl, and 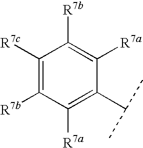
wherein each R^{7a} is independently hydrogen or halo;
each R^{7b} and R^{7c} are independently selected from the group consisting of hydrogen, halo, alkyl, acyl, acyloxy, carboxyl ester, acylamino, alkylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, alkylsulfonyl and haloalkylsulfonyl; provided that if one of R^{7b} is hydrogen and the other R^{7b} is halo, then R^{7c} and is not the same halo;
with the proviso that when m is 1, and X, Y and Z with the phenyl ring define an indole or benzimidazole, then at least one of X, Y, or Z is selected from —NR⁶— and —CR³═ where R⁶ or R³ is not hydrogen; and
with the proviso that Formula (II) is not:

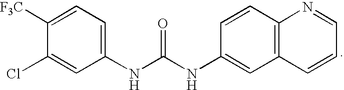

5. A compound of claim 1 or 4, wherein 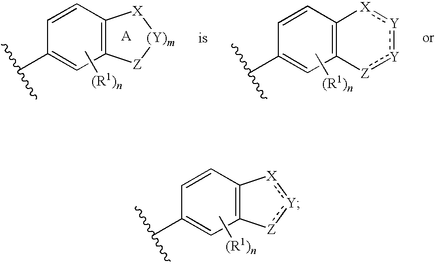
wherein
each X, Y, and Z is independently selected from the group consisting of —CR³—, —N═, —C(R⁴)(R⁵)—, —C(═O)—, —C(═S)—, —NR⁶—, —O—, —S—, —SO—, and —SO₂—; and
each [symbol] independently represents a sing bond or a double bond, provided that two double bonds are not adjacent to each other.

6. A compound of claim 5, wherein at least one of X, Y and Z is —N═ or —NR⁶—.

7. A compound of claim 1 or 4, wherein 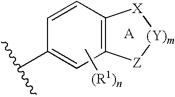
is selected from the group consisting of: 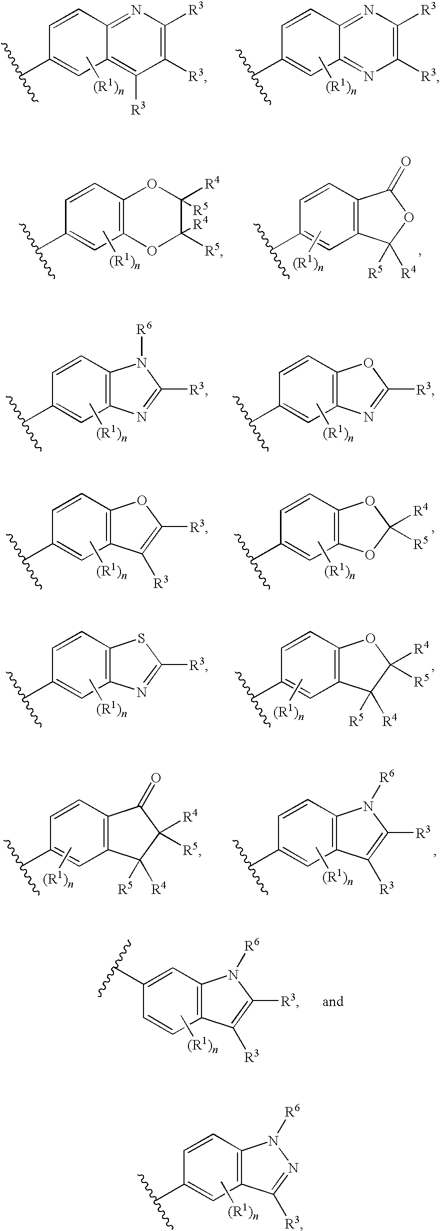
wherein each of R¹, R³, R⁴, R⁵ and R⁶ is as defined above.

8. A compound of claim 1 or 4, wherein 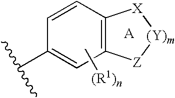
is selected from the group consisting of: 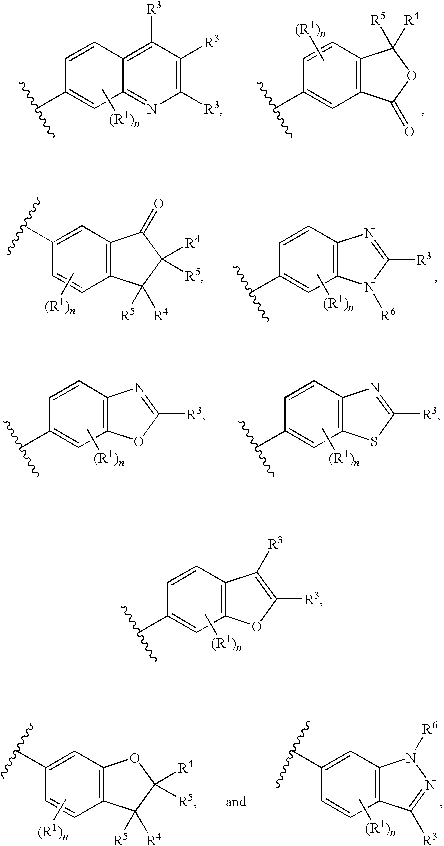
wherein each of R¹, R³, R⁴, R⁵ and R⁶ is as defined above.

9. A compound of claim 1 or 4, wherein 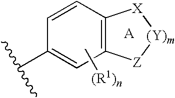
is selected from the group consisting of:

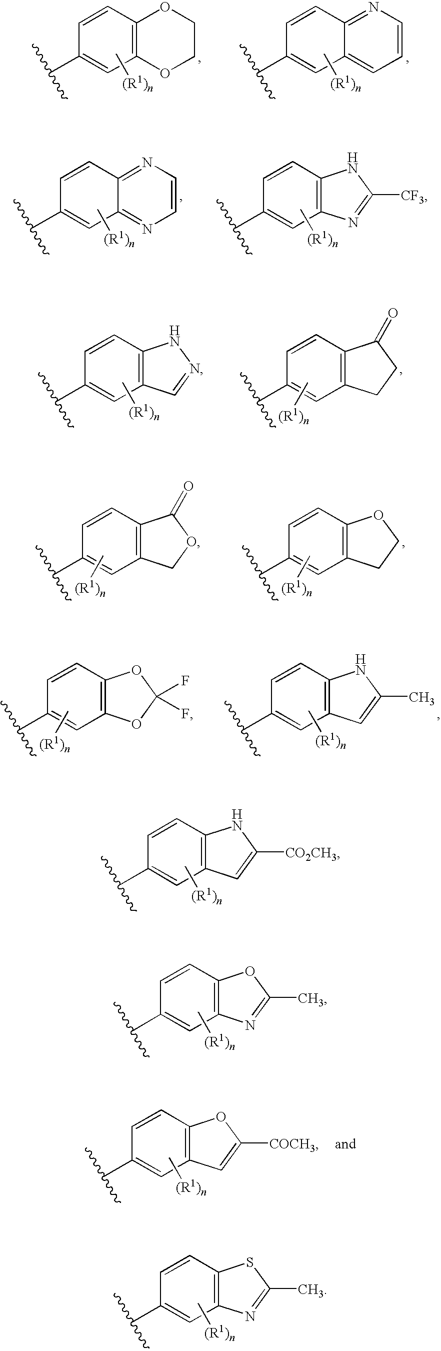

10. (canceled)

11. A compound of claim 10 wherein R^{a} is selected from the group consisting of
adamantyl,

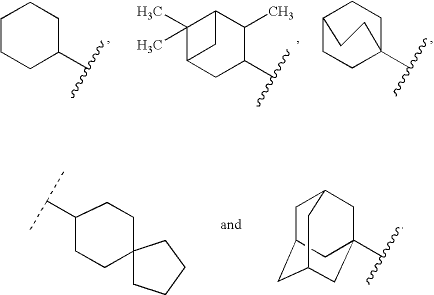

12-13. (canceled)

14. A compound of claim 13 wherein R^{a} is selected from the group consisting of 4-fluorophenyl, 3-trifluoromethylphenyl, 4-trifluoromethylphenyl, 3-trifluoromethoxyphenyl, 4-trifluoromethoxyphenyl, and 4-chlorophenyl.

15. A compound of claim 1 or 4 wherein n is 0.

16. A compound of claim 1 or 4 wherein n is 1 and R¹ is halo.

17. (canceled)

18. A compound of Formula (III) or a pharmaceutically acceptable salt thereof: 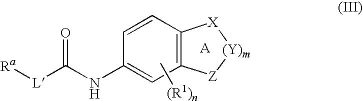
wherein:
A is an optionally substituted cycloalkyl, optionally substituted heterocyclic or optionally substituted heteroaryl ring fused with the benzene ring;
each of X, Y and Z are independently selected from the group consisting of —CR³═, —C(R⁴)(R⁵)—, —C(═O)—, —C(═S)—, —N═, —NR⁶—, —O—, —S—, —SO—, and —SO₂—, provided that at least one of X, Y and Z is selected from the group consisting of —C(═O)—, —C(═S)—, —N═, —NR⁶—, —O—, —S—, —SO—, and —SO₂—; and provided that X and Z are not both —C(═O)— and that A is not substituted with four fluoro;
wherein
each of R³, R⁴ and R⁵ is independently selected from the group consisting of R⁶, halo, alkoxy, substituted alkoxy, acyloxy, carboxyl ester, acylamino, alkylamino, aminocarbonylamino, aminocarbonyloxy, aminosulfonylamino, (substituted sulfonyl)amino, (substituted sulfonyl)aminocarbonyl, (carboxyl ester)amino;
R⁶ is independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclyl, substituted heterocyclyl, haloalkyl, acyl, aminosulfonyl, and aminocarbonyl;
m is 1, 2 or 3;
L′ is a covalent bond, —NH—, or —CR′R″— where R′ and R″ are independently H or alkyl or R′ and R″ together form a C₃-C₆ cycloalkyl ring;
each R¹ is independently selected from the group consisting of alkyl, cyano, halo, and haloalkyl;
n is 0, 1, 2, or 3; and
R^{a} is selected from the group consisting of C₆₋₁₀ cycloalkyl, substituted C₆₋₁₀ cycloalkyl, and 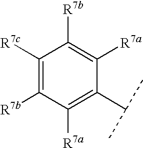
wherein each R^{7a} is independently hydrogen or halo;
each R^{7b} and R^{7c} are independently selected from the group consisting of hydrogen, halo, alkyl, acyl, acyloxy, carboxyl ester, acylamino, alkylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, alkylsulfonyl and haloalkylsulfonyl; provided that if one of R^{7b} is hydrogen and the other R^{7b} is halo, then R^{7c} and is not the same halo.

19. A compound of claim 18, wherein 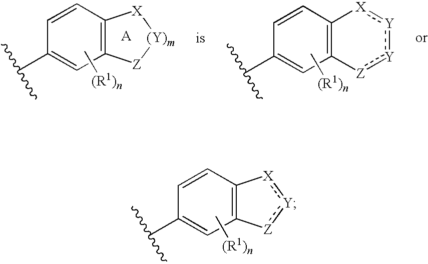
wherein
each X, Y, and Z is independently selected from the group consisting of —CR³═, —N═, —C(R⁴)(R⁵)—, —C(═O)—, —C(═S)—, —NR⁶—, —O—, —S—, —SO—, and —SO₂—; and
each [symbol] independently represents a sing bond or a double bond, provided that two double bonds are not adjacent to each other.

20. A compound of claim 19, wherein at least one of X, Y and Z is —N═ or —NR⁶—.

21. A compound of claim 18, wherein 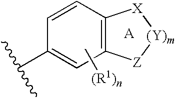
is selected from the group consisting of: 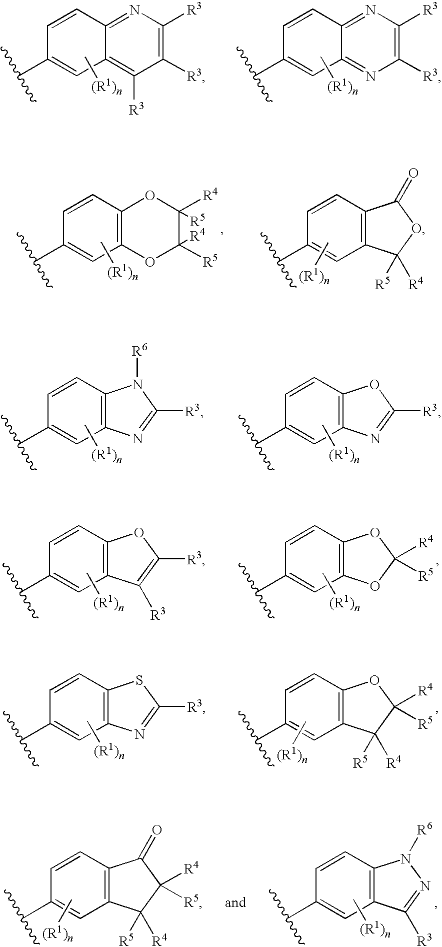
wherein each of R¹, R³, R⁴, R⁵ and R⁶ is as defined in claim 18.

22. A compound of claim 18, wherein 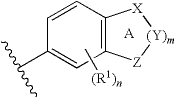
is selected from the group consisting of: 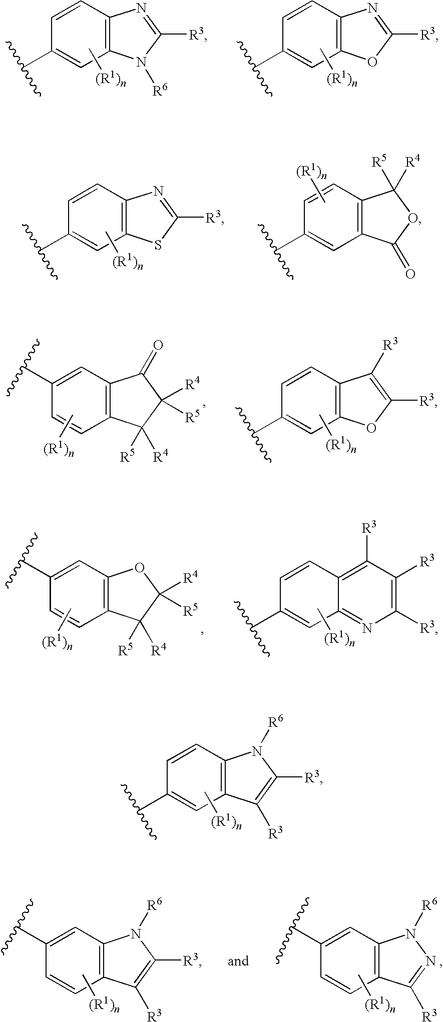
wherein each of R¹, R³, R⁴, R⁵ and R⁶ is as defined in claim 18.

23. A compound of claim 18, wherein 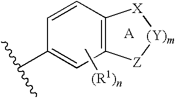
is selected from the group consisting of:

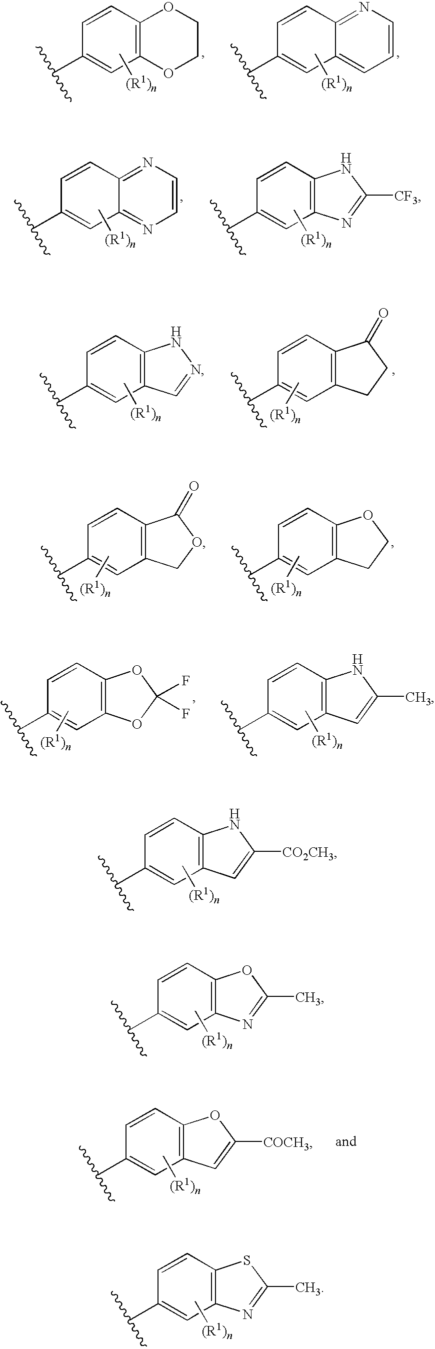

24-25. (canceled)

26. A compound of claim 24 wherein R^{a} is selected from the group consisting of
adamantyl,

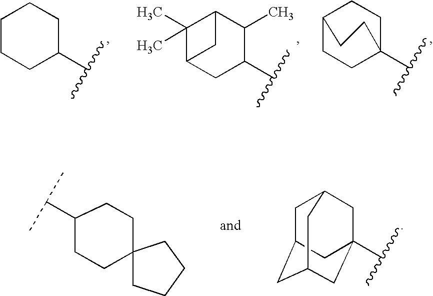

27. (canceled)

28. A compound of claim 27 wherein R^{a} is selected from the group consisting of 4-fluorophenyl, 3-trifluoromethylphenyl, 4-trifluoromethylphenyl, 3-trifluoromethoxyphenyl, 4-trifluoromethoxyphenyl, and 4-chlorophenyl.

29. A compound of claim 18 wherein n is 0.

30. A compound of claim 18 wherein n is 1 and R¹ is halo.

31. A compound of claim 30 wherein R¹ is fluoro.

32. A compound of Formula (IVa), (IVb) or (IVc), or a pharmaceutically acceptable salt thereof. 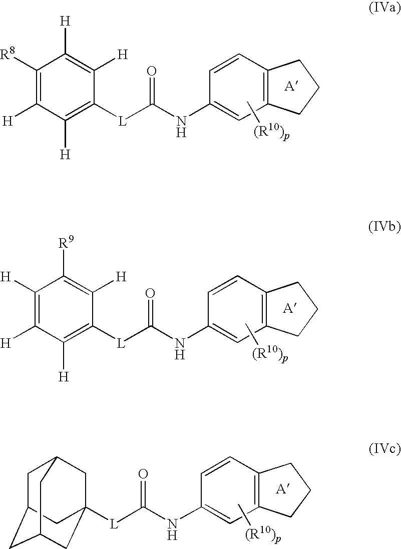
wherein
A′ is a five or six membered optionally substituted heterocyclic or optionally substituted heteroaryl ring having at least one ring nitrogen atom and is fused with the phenyl ring;
each R¹⁰ is independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclyl, substituted heterocyclyl, haloalkyl, acyl, aminosulfonyl, and aminocarbonyl, halo, alkoxy, substituted alkoxy, acyloxy, carboxyl ester, acylamino, alkylamino, aminocarbonylamino, aminocarbonyloxy, aminosulfonylamino, (substituted sulfonyl)amino, (substituted sulfonyl)aminocarbonyl, (carboxyl ester)amino;
p is 0, 1, 2, or 3;
R⁸ and R⁹ are selected from the group consisting of halo, haloalkyl, haloalkoxy, alkylthio, haloalkylthio, cyano, alkylsulfonyl, and haloalkylsulfonyl; and
L is a covalent bond, —NH—, or —CR′R″— where R′ and R″ are independently H or alkyl or R′ and R″ together form a C₃-C₆ cycloalkyl ring.

33. A compound of claim 32, wherein 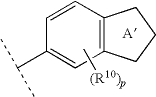
is selected from the group consisting of 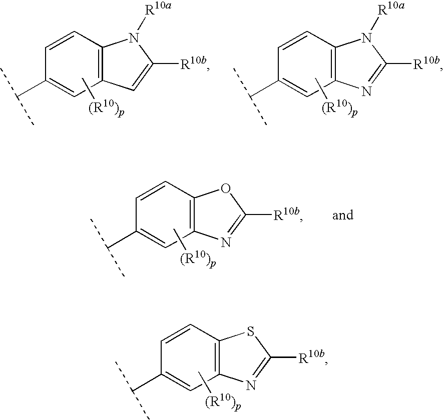
wherein
each R¹⁰ is independently selected from the group consisting of alkyl, halo, acyl, carboxyl ester and haloalkyl;
R^{10a} is hydrogen or alkyl;
R^{10b} is selected from the group consisting of hydrogen, alkyl, acyl, carboxyl ester and haloalkyl.

34. A compound of claim 32, having Formula (IVa), wherein R⁸ is selected from the group consisting of chloro, fluoro, trifluoromethoxy, and trifluoromethyl.

35. A compound of claim 32, having Formula (IVb), wherein R⁹ is selected from the group consisting of chloro, fluoro, trifluoromethoxy, and trifluoromethyl.

36. A compound of claim 1 selected from Table 3 or a pharmaceutically acceptable salt thereof:
TABLE 3

| Compound | Structure | Name |
|---|---|---|
| 1 | 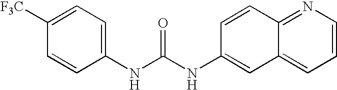 | 1-(quinolin-6-yl)-3-(4-(trifluoromethyl)phenyl)urea |
| 2 | 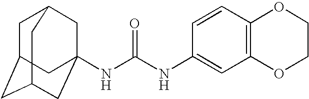 | 1-adamantan-1-yl-3-(2,3-dihydrobenzo[bl [1,4]dioxin-6-yl)urea |
| 3 | 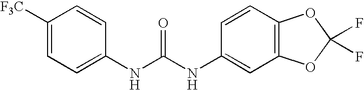 | 1-(2,2-difluorobenzo[d][1,3]dioxol-5-yl)-3-(4-(trifluoromethyl)phenyl)urea |
| 4 | 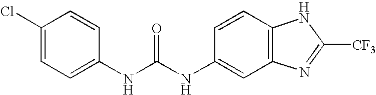 | 1-(4-chlorophenyl)-3-(2-(trifluoromethyl)-1H-benzo[d]imidazol-5-yl)urea |
| 5 | 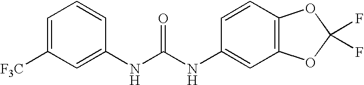 | 1-(2,2-difluorobenzo[d][1,3]dioxol-5-yl)-3-(3-(trifluoromethyl)phenyl)urea |
| 6 | 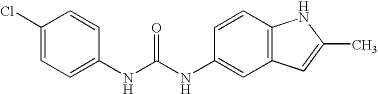 | 1-(4-chlorophenyl)-3-(2-methyl-1H-indol-5-yl)urea |
| 7 | 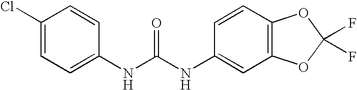 | 1-(4-chlorophenyl)-3-(2,2-difluorobenzo[d][1,3]dioxol-5-yl)urea |
| 8 | 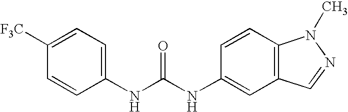 | 1-(1-methyl-1H-indazol-5-yl)-3-(4-(trifluoromethyl)phenyl)urea |
| 9 | 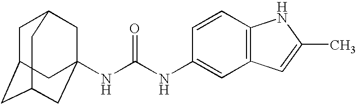 | 1-adamantan- 1-yl-3-(2-methyl-1H-indol-5-yl)urea |
| 10 | 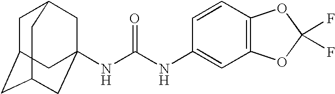 | 1-adamantan-1-yl-3-(2,2-difluorobenzo[d][1,3]dioxol-5-yl)-urea |
| 11 | 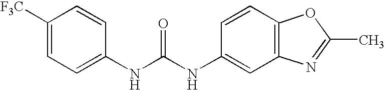 | 1-(2-methylbenzo[d]oxazol-5-yl)-3-(4-(trifluoromethyl)phenyl)urea |
| 12 | 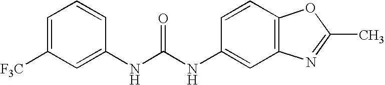 | 1-(2-methylbenzo[d]oxazol-5-yl)-3-(3-(trifluoromethyl)phenyl)urea |
| 13 | 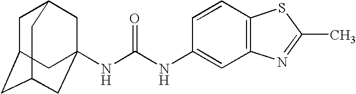 | 1-adamantan-1-yl-3-(2-methylbenzo[d]thiazol-5-yl)urea |
| 14 | 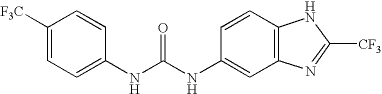 | 1-(2-(trifluoromethyl)-1H-benzo[d]imidazol-5-yl)-3-(4-(trifluoromethyl)phenyl)urea |
| 15 | 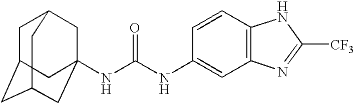 | 1-adamantan-1-yl-3-(2-(trifluoromethyl)-1H-benzo[d]imidazol-5-yl)urea |
| 16 | 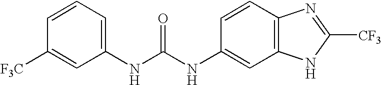 | 1-(2-(trifluoromethyl)-1H-benzo[d]imidazol-5-yl)-3-(3-(trifluoromethyl)phenyl)urea |
| 17 | 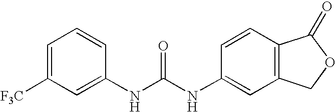 | 1-(1-oxo-1,3-dihydroisobenzofuran-5-yl)-3-(3-(trifluoromethyl)phenyl)urea |
| 18 | 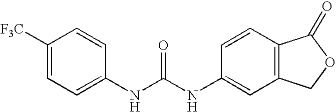 | 1-(1-oxo-1,3-dihydroisobenzofuran-5-yl)-3-(4-(trifluoromethyl)phenyl)urea |

37. A pharmaceutical composition comprising a pharmaceutically acceptable carrier and a therapeutically effective amount of a compound of any one of claims 1, 18, and 36 or a pharmaceutically acceptable salt thereof.

38. A method for treating a soluble expoxide hydrolase mediated disease, said method comprising administering to a patient a compound of any one of claims 1, 18, and 36 or a pharmaceutically acceptable salt thereof.

39. (canceled)

40. A method for inhibiting a soluble epoxide hydrolase, comprising contacting the soluble epoxide hydrolase with an effective amount of a compound of any one of claims 1, 18, and 36 or a pharmaceutically acceptable salt thereof.
